(19) United States
(12) Patent Application Publication
(10) Pub. No.: US 2017/0014360 A1
(43) Pub. Date: Jan. 19, 2017

(54) METHODS OF TREATING CANCER USING RAD51 SMALL MOLECULE STIMULATORS

(71) Applicant: The University of Chicago, Chicago, IL (US)

(72) Inventors: Phillip P. CONNELL, Chicago, IL (US); Douglas K. BISHOP, Chicago, IL (US); Brian BUDKE, Chicago, IL (US); Ralph WEICHSELBAUM, Chicago, IL (US)

(21) Appl. No.: 15/124,283

(22) PCT Filed: Mar. 10, 2015
(86) PCT No.: PCT/US2015/019578

§ 371 Date: Sep. 7, 2016

Related U.S. Application Data
(60) Provisional application No. 61/950,689, filed on Mar. 10, 2014.

(51) Int. Cl. A61K 31/18; A61K 31/415; A61K 45/06; C07D 231/14; C07C 311/16; C07C 311/21; C07D 213/40; A61K 31/4406; A61K 31/4709

(52) U.S. Cl. CPC A61K 31/18; A61K 31/4406; A61K 31/415; A61K 45/06; A61K 31/4709; C07C 311/16; C07C 311/21; C07D 213/40; C07D 231/14

(57) ABSTRACT

Methods of killing or inhibiting the growth cancer cells and tumors and of treating cancer by administering compounds that stimulate the activity of RAD51. Cells overexpressing RAD51 or with other imbalances in homologous recombination machinery are particularly susceptible targets of RAD51 stimulators.

### CROSS-REFERENCE TO RELATED APPLICATIONS

This application claims the benefit of priority of U.S. Provisional Patent Application No. 61/950,689, filed Mar. 10, 2014, which is hereby incorporated by reference in its entirety.

### NOTICE OF GOVERNMENT RIGHTS

This invention was made with government support under CA142642-02 2010-2015 awarded by the National Institutes of Health. The government has certain rights in the invention.

### BACKGROUND OF THE INVENTION

A. Field of the Invention

The present invention relates generally to the fields of biochemistry, cell biology, and oncology. More specifically, it concerns methods for killing or inhibiting cancer cells by stimulating RAD51 protein activity.

B. Description of Related Art

Homologous recombination (HR) is an essential process that serves multiple roles including the repair of DNA double strand breaks (DSBs). HR utilizes an undamaged sister chromatid as a template to guide the repair of DSBs, thereby leading to error-free repair. HR also promotes cellular recovery from replication-blocking lesions or collapsed replication forks. Because of these repair activities, cells that harbor HR defects exhibit profound sensitivities to several classes of chemotherapeutics, including PARP inhibitors and inter-strand DNA cross-linkers that interfere with DNA replication or replication-associated DNA repair (Tebbs, et al., 1995; Liu, et al., 1998; Takata, et al., 2001).

RAD51 is a highly conserved DNA binding protein that is central to HR. While RAD51 generally plays a protective role against DNA damage in cells, it can also be responsible for processes detrimental to cell growth and survival if it is expressed at high levels or if function of the RAD54 translocases RAD54B or RAD54L is diminished. Several cancers and cell lines have imbalances in the activity levels of these proteins, making them potentially susceptible to therapies that target RAD51 activity.

### SUMMARY OF THE INVENTION

The present invention provides methods of killing or inhibiting cancer cells using RAD51 stimulators that further enhance the toxic effect of imbalances in the expression and activity levels of RAD51 and RAD54 family proteins in cancer cells.

Disclosed is a method of killing or inhibiting the growth of cells comprising contacting the cells with a composition comprising an amount of a RAD51 stimulator effective to kill or inhibit the growth of the cells. In some embodiments, the RAD51 stimulator is a compound having the formula (VIIa):

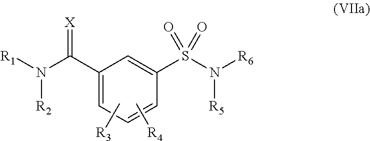

wherein: R₁ is hydrogen, alkyl, aryl or aralkyl; R₂ is alkyl, aryl or aralkyl; X is O or S; R₃ is hydrogen, halogen, alkyl or alkoxy; R₄ is hydrogen, halogen, alkyl or alkoxy; R₅ is hydrogen, alkyl, aryl, or aralkyl; and R₆ is hydrogen, alkyl, aryl or aralkyl. In some embodiments, R₃ is substituted at the 4 position and R₄ is substituted at the 6 position. In some embodiments, halogen of R₃ and R₄ are both chloride or bromide. In some embodiments, R₃ is hydrogen and R₄ is either chloride or bromide. In some embodiments, R₄ is hydrogen and R₃ is either chloride or bromide. In some embodiments, R₃ is hydrogen and R₄ is methyl. In some embodiments, R₃ is hydrogen and R₄ is methoxy. In some embodiments, R₁ is:

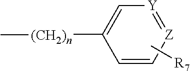

wherein: n is 0-6; Y is C or N; Z is C or N; and R₇ is hydrogen, halogen, alkyl, alkoxy or carboxy. In some embodiments, Y and Z are both C and R₇ is substituted at the 2 or 4 position. In some embodiments, R₇ is a chloride or bromide. In some embodiments, R₇ is methyl. In some embodiments, R₇ is methoxy. In some embodiments, R₆ is:

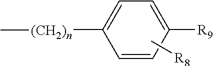

wherein: n is 0-6; R₈ is hydrogen or alkyl; and R₉ is hydrogen, halogen or alkyl. In some embodiments, R₈ is methyl and substituted at the 2 or 3 position. In some embodiments, R₉ is methyl. In some embodiments, R₈ is hydrogen and the halogen of R₉ is bromide. In some embodiments, the RAD51 stimulator is a compound having the formula (VIIb):

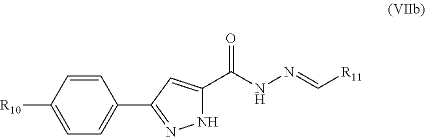

wherein: R₁₀ is halogen or alkoxy; and R₁₁ is aryl. In some embodiments, R₁₀ is chloride. In some embodiments, R₁₀ is methoxy or ethoxy. In some embodiments, R₁₁ is:

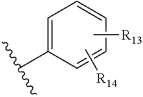

wherein: R₁₃ is hydroxyl or methoxy; and R₁₄ is hydroxyl. In some embodiments, R₁₃ is substituted at the 4 position and R₁₄ is substituted at the 2 or 3 position. In some embodiments, the RAD51 stimulator is a compound having the formula (VIIc):

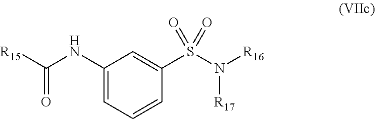

wherein: R₁₅ is C₁-C₁₀ alkyl; R₁₆ is aryl; and R₁₇ is hydrogen. In some embodiments, R₁₅ is iso-butyl. In some embodiments, R₁₅ is 4-bromophenyl. In some embodiments, the RAD51 stimulator is a compound having the following formula:

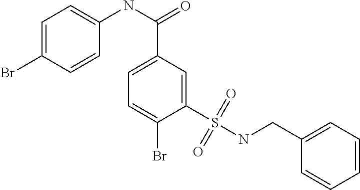 [SMILES: O=C(Nc1ccc(Br)cc1)c1ccc(Br)c(S(=O)(=O)NCc2ccccc2)c1]

In some embodiments, the cells that are killed or the growth of which are inhibited have an increased sensitivity to the RAD51 stimulator relative to a control level of sensitivity. In some embodiments, the cells are determined to have an increased sensitivity to the RAD51 stimulator relative to a control level of sensitivity. In some embodiments, the cells express an increased level of RAD51 relative to a control level. In some embodiments, the cells have been determined to express an increased level of RAD51 relative to a control level. In some embodiments, the cells have a decreased activity or expression level of RAD54B, RAD54L, or both, relative to a control level. In some embodiments, the cells have been determined to have a decreased activity or expression level of RAD54B, RAD54L, or both, relative to a control level. In some embodiments, the cells are in cell culture. In some embodiments, the cells are in a patient's body. In some embodiments, the cells are cancer cells. In some embodiments, the cells are in a tumor. In some embodiments, the composition with which the cells are contacted comprises 20 to 80 μM of RAD51 stimulator. In some embodiments, the cells are not exposed to a substantial amount of any DNA damaging agent.

In some embodiments, a method of killing or inhibiting the growth of cells comprises contacting the cells with a composition comprising an amount of a RAD51 stimulator and a DNA damaging agent. In some aspects of the invention, a method of killing or inhibiting the growth of cells comprising contacting the cells with a RAD 51 stimulator and a RAD54 inhibitor. In some embodiments, the RAD54 inhibitor is streptonigrin. In further embodiments, the method comprises contacting the cells with a RAD 51 stimulator, a RAD54 inhibitor, and a DNA damaging agent.

Also disclosed is a method of selectively killing or inhibiting the growth of cancer cells in a subject comprising administering to the subject a pharmaceutically acceptable composition comprising an amount of RAD51 stimulator effective to selectively kill or inhibit the growth of the cancer cells. In some embodiments, the RAD51 stimulator is a compound having the formula (VIIa):

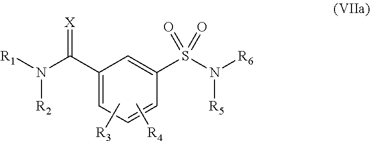

wherein: R₁ is hydrogen, alkyl, aryl or aralkyl; R₂ is alkyl, aryl or aralkyl; X is O or S; R₃ is hydrogen, halogen, alkyl or alkoxy; R₄ is hydrogen, halogen, alkyl or alkoxy; R₅ is hydrogen, alkyl, aryl, or aralkyl; and R₆ is hydrogen, alkyl, aryl or aralkyl. In some embodiments, R₃ is substituted at the 4 position and R₄ is substituted at the 6 position. In some embodiments, halogen of R₃ and R₄ are both chloride or bromide. In some embodiments, R₃ is hydrogen and R₄ is either chloride or bromide. In some embodiments, R₄ is hydrogen and R₃ is either chloride or bromide. In some embodiments, R₃ is hydrogen and R₄ is methyl. In some embodiments, R₃ is hydrogen and R₄ is methoxy. In some embodiments, R₁ is:

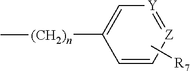

wherein: n is 0-6; Y is C or N; Z is C or N; and R₇ is hydrogen, halogen, alkyl, alkoxy or carboxy. In some embodiments, Y and Z are both C and R₇ is substituted at the 2 or 4 position. In some embodiments, R₇ is a chloride or bromide. In some embodiments, R₇ is methyl. In some embodiments, R₇ is methoxy. In some embodiments, R₆ is:

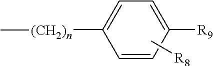

wherein: n is 0-6; R₈ is hydrogen or alkyl; and R₉ is hydrogen, halogen or alkyl. In some embodiments, R₈ is methyl and substituted at the 2 or 3 position. In some embodiments, R₉ is methyl. In some embodiments, R₈ is hydrogen and the halogen of R₉ is bromide. In some embodiments, the RAD51 stimulator is a compound having the formula (VIIb):

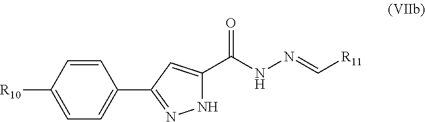

wherein: R₁₀ is halogen or alkoxy; and R₁₁ is aryl. In some embodiments, R₁₀ is chloride. In some embodiments, R₁₀ is methoxy or ethoxy. In some embodiments, R₁₁ is:

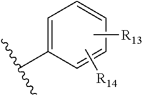

wherein: R₁₃ is hydroxyl or methoxy; and R₁₄ is hydroxyl. In some embodiments, R₁₃ is substituted at the 4 position and R₁₄ is substituted at the 2 or 3 position. In some embodiments, the RAD51 stimulator is a compound having the formula (VIIc):

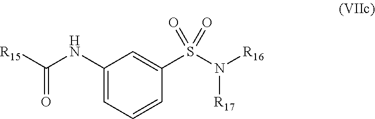

wherein: R₁₅ is C₁-C₁₀ alkyl; R₁₆ is aryl; and R₁₇ is hydrogen. In some embodiments, R₁₅ is iso-butyl. In some embodiments, R₁₅ is 4-bromophenyl. In some embodiments, the RAD51 stimulator is a compound having the following formula:

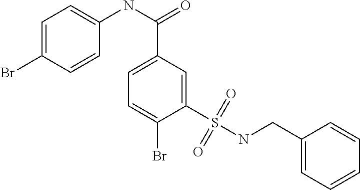 [SMILES: O=C(Nc1ccc(Br)cc1)c1ccc(Br)c(S(=O)(=O)NCc2ccccc2)c1]

In some embodiments, the subject has cancer of the lung, liver, skin, eye, brain, gum, tongue, hematopoietic system or blood, head, neck, breast, pancreas, prostate, kidney, bone, testicles, ovary, cervix, gastrointestinal tract, lymph system, small intestine, colon, or bladder. In some embodiments, the cancer cells are in a tumor. In some embodiments, the composition comprises an amount of RAD51 stimulator effective to shrink or inhibit the growth of the tumor. In some embodiments, the cancer cells have an increased sensitivity to the RAD51 stimulator relative to a control level of sensitivity. In some embodiments, the cancer cells have been determined to have an increased sensitivity to the RAD51 stimulator relative to a control level of sensitivity. In some embodiments, the cancer cells express an increased level of RAD51 relative to a control level. In some embodiments, the cancer cells have been determined to express an increased level of RAD51 relative to a control level. In some embodiments, the cancer cells have a decreased activity or expression level of RAD54B, RAD54L, or both, relative to a control level. In some embodiments, the cancer cells have been determined to have a decreased activity or expression level of RAD54B, RAD54L, or both, relative to a control level. In some embodiments, the subject is administered a dose of 50 to 150 mg/kg of the RAD51 stimulator. In some embodiments, the subject is administered a dose of 110 mg/kg. In some embodiments, the RAD51 stimulator is present in the blood of the subject in a concentration of 250 to 350 μM. In some embodiments, the RAD51 stimulator is present in the blood of the subject in a concentration of 300 μM. In some embodiments, the subject is not administered a substantial amount of any DNA damaging agent within three days of administering to the subject the RAD51 stimulator. In some embodiments, the subject is not exposed to a substantial amount of any DNA damaging agent within seven days of administering the RAD51 stimulator to the subject. In some embodiments, the subject is not exposed to a substantial amount of any DNA damaging agent after administering the RAD51 stimulator to the subject. In some embodiments, the subject is not administered a DNA damaging agent as part of a combination therapy with the RAD51 stimulator. In other embodiments, the subject is administered a DNA damaging agent as part of a combination therapy with the RAD51 stimulator. In some embodiments, a RAD51 stimulator is administered concurrently with a DNA damaging agent. In some aspects of the invention, a RAD51 stimulator is administered after administration of a DNA damaging agent. In other embodiments, the DNA damaging agent is administered after administering a RAD51 stimulator. The DNA damaging agent may be administered immediately after, or anywhere from immediately after to 30 days after administration of a RAD51 stimulator. In some embodiments, the subject is administered a RAD54 inhibitor as part of a combination therapy with the RAD51 stimulator. In some embodiments, the RAD54 inhibitor is streptonigrin. In further embodiments, the subject is administered a combination therapy of a RAD51 stimulator, a RAD54 inhibitor, and a DNA damaging agent. In some embodiments, the RAD51 stimulator is administered to the subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularly, mucosally, intrapericardially, intraumbilically, intraocularally, orally, topically, locally, by inhalation, by injection, by infusion, by continuous infusion, by localized perfusion bathing target cells directly, via a catheter, or via a lavage. In some embodiments, the RAD51 stimulator is administered to the patient multiple times. In some embodiments, the subject is administered an additional cancer therapy.

Also disclosed is method of treating cancer in a patient comprising administering an effective amount of a RAD51 stimulator after determining that the cancer has increased sensitivity to the RAD51 stimulator relative to a control level of sensitivity. In some embodiments, the method further comprises measuring the expression or activity level of RAD51, RAD54B, and/or RAD54L, in the cancer and comparing it to a control level. It is contemplated that any of the RAD51 stimulators described for use in any of the methods above can also be used to perform this method.

An “effective amount” of a compound or composition, generally, is defined as that amount sufficient to detectably and repeatedly achieve the stated desired result, for example, to ameliorate, reduce, minimize or limit the extent of the disease or its symptoms or to increase, stimulate, or promote a desirable physiological response, such as homologous recombination. More rigorous definitions may apply, including elimination, eradication or cure of disease.

It is contemplated that in certain embodiments, a cell is a human cell and the subject or patient is a human patient. In other embodiments, a cell is a mammalian cell and the subject or patient is a mammalian patient. In some embodiments, a cell is a Drosophila cell and the subject or patient is a Drosophila patient. It will be understood that different mammals have their own RAD51 protein that would be a homolog of the human protein. In certain other embodiments, the cell is a eukaryotic cell, while in other embodiments, the cell is a prokaryotic cell and a RAD51 protein homolog or analog is the protein that is stimulated. In specific embodiments, a cell may be a sex cell, while in others, the cell is a somatic cell. In particular embodiments, cells used in methods of the invention may be from a cell line. In certain embodiments, the cell is a cell from or in any organism described herein. Moreover, in some embodiments the cell is a cancer cell, while in other embodiments a cell is non-cancerous or normal. In some cases, a cancer cell is resistant to chemotherapy or radiation. Furthermore, it is contemplated that a cell can be in a patient. Additionally, a cell may be an embryonic stem (ES) cell, such as a murine ES cell, which are used for generating knockout mice. Alternatively, cells may be murine cells that are used for generating a transgenic mouse. Other transgenic animals can be generated using a particular animals cells in the context of methods of the invention.

The small molecules described herein typically contain an aryl group. Accordingly, in certain embodiments, compounds comprising one or more aryl groups are contemplated. The aryl groups may be substituted by any substituent known to those of skill in the art (e.g., H, amino, nitro, halo, mercapto, cyano, azido, silyl, hydroxy, alkyl, alkenyl, alkynyl, aryl, aralkyl, alkoxy, alkenoxy, alkynyloxy, aryloxy, acyloxy, alkylamino, alkenylamino, alkynylamino, arylamino, aralkylamino, amido, alkylthio, alkenylthio, alkynylthio, arylthio, aralkylthio, acylthio, alkylsilyl, phosphonate, phosphinate, or any combination thereof). Subsets of these substituent groups at any aryl position are also contemplated (e.g., compounds of formula I, II, III, IV, V, VI VII, or any combination thereof). In certain embodiments, the small molecules are any one or more of the specific chemical compounds whose structures are shown herein.

A “disease” is defined as a pathological condition of a body part, an organ, or a system resulting from any cause, such as infection, genetic defect, or environmental stress. A “health-related condition” is defined herein to refer to a condition of a body part, an organ, or a system that may not be pathological, but for which treatment is sought. Examples include conditions for which cosmetic therapy is sought, such as skin wrinkling, skin blemishes, and the like. The disease can be any disease, and non-limiting examples include hyperproliferative diseases such as cancer and premalignant lesions, wounds, and infections.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset of a disease or health-related condition.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, the methods and systems of the present invention that “comprises,” “has,” “includes” or “contains” one or more elements possesses those one or more elements, but is not limited to possessing only those one or more elements. Likewise, an element of a method or system of the present invention that “comprises,” “has,” “includes” or “contains” one or more features possesses those one or more features, but is not limited to possessing only those one or more features.

Any method or system of the present invention can consist of or consist essentially of—rather than comprise/include/contain/have—any of the described elements and/or features and/or steps. Thus, in any of the claims, the term “consisting of” or “consisting essentially of” can be substituted for any of the open-ended linking verbs recited above, in order to change the scope of a given claim from what it would otherwise be using the open-ended linking verb.

Throughout this application, the term “about” is used to indicate that a value includes the standard deviation of error for the device and/or method being employed to determine the value.

The term “substantially” is defined as being largely but not necessarily wholly what is specified (and include wholly what is specified) as understood by one of ordinary skill in the art. In any disclosed embodiment, the term “substantially” may be substituted with “within [a percentage] of” what is specified, where the percentage includes 0.1, 1, 5, and 10 percent.

As used herein, in the specification, “a” or “an” may mean one or more, unless clearly indicated otherwise. As used herein, in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers.

FIG. 1. Low expression levels of the RAD54 translocase proteins are significantly associated with RS-1 sensitivity in cell lines. Protein levels from Table 1 are plotted as a function of RS-1 sensitivity. Translocase protein expression level is defined as RAD54B+RAD54L. The displayed trend line is the result of linear regression analysis.

FIG. 2. Forced overexpression of RAD51 expression levels sensitizes cells to RS-1. HT1080 cells carrying a doxycycline-repressible RAD51 transgene were pre-treated with varying levels of doxycycline for 24 hours. Western blot shows RAD51 protein levels in upper panel. In lower panel, cells were subsequently incubated for 24 hours in media containing varying concentrations of RS-1. Cells were then allowed to grow in drug-free media for an additional 6 days, and indicated doxycycline concentrations were maintained throughout the entire experiment. Average survival for each condition is normalized to the 0 μM RS-1 control of that condition. Quantifications of western blots are displayed in FIG. 9.

FIGS. 3A-3B. Knockdown of RAD51 in PC3 improves viability and protects cells from the toxicity of RS-1. PC3 cells were treated with various concentrations of RAD51 siRNA for 48 hours. FIG. 3A: Western blot shows RAD51 protein levels in upper panel. Following RNAi, cells were allowed to grow for 7 days and assayed for viability in the absence of additional treatment. FIG. 3B: Following RNAi, cells were incubated for 24 hours in media containing varying concentrations of RS-1. Cells were then allowed to grow in drug-free media for an additional 6 days. Average survival for each condition is normalized to the 0 μM RS-1 control of that condition. Quantifications of western blots are displayed in FIGS. 9A-9C.

FIG. 4. Knockdown of RAD54L and RAD54B sensitizes cancer cells to the toxicity of RS-1. PC3 cells were treated with siRNA against RAD54L, RAD54B, both, or a non-silencing (NS) control for 48 hours. Following RNAi, cells were subsequently incubated for 24 hours in media containing varying concentrations of RS-1. Cells were then allowed to grow in RS-1-free media for an additional 6 days. Average survival for each condition is normalized to the 0 μM RS-1 control of that condition. Quantifications of western blots are displayed in FIGS. 9A-9C.

FIG. 5. RS-1 stimulates the binding of RAD51 to both ssDNA and dsDNA. Various concentrations of RS-1 were incubated with purified hRAD51 protein and a fluorescently tagged DNA substrate, consisting of either a ssDNA oligonucleotide (DHD162-CD-CF) or a dsDNA double hairpin (DHD162). Binding of RAD51 to DNA was measured as a function of fluorescence polarization of the tag, as described in the methods section.

FIGS. 6A-6B. RS-1 generates microscopically visible RAD51 complexes in undamaged PC3 nuclei, but not in non-immortalized MRC-5 nuclei. FIG. 6A: Cells were grown on cover slips, incubated for 6 hours in media containing 60 μM RS-1. In the 8 Gy radiation control condition, cells were irradiated 6 hours before harvest. Cells were subsequently indirectly immunostained. Representative images of key conditions are displayed with RAD51 displayed in green and RPA displayed in red. FIG. 6B: Fifty randomly selected nuclei per treatment group were examined and discrete foci were quantified. The displayed p values were calculated using the fisher's exact test.

FIG. 7. RS-1 generates anti-tumor responses in a mouse xenograft tumor model. PC3 tumors were induced in the hind limbs of athymic nude mice. Mice were then randomized into two treatment groups. Starting on day 0, mice then received 5 daily intra-peritoneal injections with either RS-1 (110 mg/kg) or vehicle alone control. Median tumor volume is plotted, normalized to the starting tumor volume on day 0.

FIGS. 8A-8E. Structures of RAD51 stimulators.

FIGS. 9A-9C. Quantifications of western blots are displayed. Quantifications of protein levels for: FIG. 9A: RAD51 levels in HT1080 cells carrying a doxycycline-repressible RAD51 transgene (from the western blot in FIG. 2), FIG. 9B: RAD51 levels in PC3 cells (from the western blot in FIG. 3), FIG. 9C: RAD54B and RAD54L levels in PC3 cells (from the western blot in FIG. 4).

### DESCRIPTION OF ILLUSTRATIVE EMBODIMENTS

The present invention provides methods of treating cancer, shrinking or inhibiting the growth of tumors, and killing or inhibiting the growth of cells, including cancer cells, using small molecules that directly stimulate, enhance, or increase the activity of RAD51. As discussed above, elevated expression of RAD51 or decreased activity of RAD54 family proteins causes cells to be sensitive to RAD51 stimulators. The present invention takes advantage of this sensitivity by using RAD51 stimulators to kill or inhibit sensitive cells. In addition, a RAD51 stimulator may be used in conjunction with a RAD54 inhibitor and/or a DNA damaging agent.

### A. RAD51 PROTEIN

The methods of the present invention use small molecules that directly stimulate the activity of RAD51 protein. “Direct” stimulation refers to increase in the activity of RAD51 molecules themselves, as contrasted with achieving an increase in RAD51 activity via increased expression of the protein.

RAD51 is a highly conserved protein that is central to HR. HR events involve 5′ to 3′ nuclease processing of DNA ends that generates 3′ single-stranded DNA (ssDNA) tails at the sites of damaged DNA. These tracks of ssDNA rapidly become coated bound by single strand DNA-binding protein RPA. RPA is ultimately displaced from the ssDNA by oligomerization of RAD51 protein on ssDNA, wherein protomers of RAD51 oligomerize into a helical, right-handed nucleoprotein filament. The ability of RAD51 to displace RPA on ssDNA in cells requires several mediator proteins, which include BRCA2, RAD52, the RAD51 paralog complexes, and other proteins (Thompson & Schild, 2001). Cells that harbor defects in mediator proteins exhibit low HR efficiency, and the overexpression of RAD51 protein can partially circumvent deficient mediator functions (Takata, et al., 2001; Martin, et al., 2007; Brown & Holt, 2009; Lee, et al., 2009).

RAD51 overexpression to modestly elevated levels can stimulate HR activity, at least in some systems (Vispe, et al., 1998; Slupianek, et al., 2001; Bello, et al., 2002; Hansen, et al., 2003). By contrast, RAD51 overexpression to much higher levels tends to generate negative consequences for cells, in terms of both lower HR efficiency and reduced viability (Martin, et al., 2007; Kim, et al., 2001; Flygare, et al., 2001). For example, RAD51 protein expression was experimentally increased by >10-fold using HT1080 cells that carry a repressible RAD51 transgene, and this resulted in slower growth rate, G2 arrest, and apoptosis (Flygare, et al., 2001). In another example, forced overexpression of RAD51 lead to the formation of aberrant homology-mediated repair products and chromosomal translocations (Richardson, et al., 2004).

Under the normal conditions of proper HR repair, RAD51 is known to accumulate into sub-nuclear foci at sites of ssDNA that are undergoing repair (Bishop, 1994; Haaf, et al., 1995). However, some human cancer cell lines that overexpress RAD51 to very high levels exhibit nuclear foci of RAD51 in the absence of exogenous DNA damage, while such non-damage induced foci are far less prominent in nonmalignant cells (Raderschall, et al., 2002). Therefore the toxicity associated with very high levels of RAD51 expression may be related to RAD51 complexes that accumulate on undamaged double-stranded DNA (dsDNA) (Shah, et al., 2010).

These findings have important implications to human malignancies, since RAD51 protein is commonly overexpressed in human cancers and cell lines (Klein, 2008; Maacke et al., 2000a; Maacke et al., 2000b; Han et al., 2002; Henning and Sturzbecher, 2003; Yoshikawa et al., 2000; Qiao et al., 2005; Raderschall et al., 2002; Russell et al., 2003; Hansen et al., 2003). This overexpression seems largely due to transcriptional up-regulation, given that the RAD51 promoter is activated an average of 840-fold (with a maximum difference of 12,500-fold) in a wide range of cancer cell lines, relative to normal human fibroblasts (Hine, et al., 2008). Human tumors with the highest levels of RAD51 overexpression tend to exhibit aggressive pathologic features (Maacke, et al., 2000; Mitra, et al., 2009), and patients accordingly experience relatively poor outcomes (Connell, et al., 2006; Qiao, et al., 2005; Takenaka, et al., 2007). Taken together, these observations indicate that RAD51 overexpression may be a common mechanism leading to genomic instability, which in turn fuels malignant progression of human cancers. Analysis of tumor cells containing high levels of non-damage-associated RAD51 complexes indicates that defects in chromosome segregation underlie this instability (Mason, et al., 2013).

RAD51 over-expression is particularly dramatic in the case of pancreatic cancer. Han et. al. (2002) performed a cDNA microarray analysis comparing pancreatic cancer cells lines to normal pancreatic cells; RAD51 was among the 30 most over-expressed genes in this analysis. This result was confirmed with an immunohistochemical (IHC) analysis showing strong RAD51 staining in 71.8% of malignant pancreatic tumors in humans (Han et al., 2002). A similar study of 47 human pancreatic tumor tissue specimens showed RAD51 overexpression in 66% of tumors (Maacke et al., 2000b). In fact, RAD51 overexpression is so great that 7% of pancreatic cancer patients generate auto-antibodies to RAD51, which can be detected in their sera (Maacke et al., 2002).

Some RAD51 stimulators affect RAD51 filament formation, which, as discussed above, is a critical step in the initiation of HR repair. Biochemical studies have shown that RAD51 protein assembles into filaments readily on sites of single stranded DNA (ssDNA). In vitro filament formation is magnesium and ATP dependent, and requires a concentration of RAD51 protein of approximately 250 nM. This reaction also demonstrates cooperativity, such that a threshold level of RAD51 binding to ssDNA will stimulate further filament formation ((Zaitseva et al., 1999; Shinohara et al., 1992).

### B. RAD54 PROTEINS

RAD51-mediated toxicity can result not only from RAD51 overexpression, but also from decreased expression or activity of the RAD54 family translocases RAD54B and RAD54L. As discussed above, the toxicity associated with very high levels of RAD51 expression may be related to RAD51 complexes that accumulate on undamaged double-stranded DNA (dsDNA) (Shah, et al., 2010). These damage-independent RAD51 complexes can be ameliorated, at least in part, by Swi2/Snf2-related translocases. For example, yeast Rad54 protein was shown to dissociate RAD51 nucleoprotein filaments formed on dsDNA in biochemical systems (Solinger, et al., 2002). Additional work in yeast has demonstrated that RAD51 accumulates spontaneously on chromatin when a set of three partially-redundant DNA translocases (Rad54, Rdh54, or Uls1) are absent. This cytologic observation coincides with slower cell growth and elevated genomic instability (Shah, et al., 2010). Translocase depletion can also result in accumulation of non-damage-associated RAD51 complexes bound to DNA (Mason, et al., 2013). Therefore, the propensity for cancer cells to form toxic RAD51 complexes likely reflects an imbalance between RAD51 protein concentration and the combined activities of RAD54 family translocases.

Mutations in RAD54 family proteins are associated with cancer. For example, homozygous mutations at highly conserved positions of RAD54B have been observed in human primary lymphoma and colon cancer (Hiramoto et al., 1999), and SNPs in RAD54B and RAD54L are significantly associated with risk of esophageal squamous cell carcinoma and gastric cancer, respectively (Li et al., 2013).

### C. CANCER AND DNA DAMAGING AGENTS

Cancer cells that may be treated by methods and compositions of the invention include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The term “DNA damaging agent” refers to any agent that directly or indirectly damages DNA for which homologous recombination could repair the damage. Specific examples of DNA-damaging agents include alkylating agents, nitrosoureas, anti-metabolites, plant alkaloids, plant extracts and radioisotopes. Specific examples of agents also include DNA-damaging drugs, for example, 5-fluorouracil (5-FU), capecitabine, S-1 (Tegafur, 5-chloro-2,4-dihydroxypyridine and oxonic acid), 5-ethynyluracil, arabinosyl cytosine (ara-C), 5-azacytidine (5-AC), 2′,2′-difluoro-2′-deoxycytidine (dFdC), purine antimetabolites (mercaptopurine, azathiopurine, thioguanine), gemcitabine hydrochlorine (Gemzar), pentostatin, allopurinol, 2-fluoro-arabinosyl-adenine (2F-ara-A), hydroxyurea, sulfur mustard (bischloroetyhylsulfide), mechlorethamine, melphalan, chlorambucil, cyclophosphamide, ifosfamide, thiotepa, AZQ, mitomycin C, dianhydrogalactitol, dibromoducitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubicin (Cerubicine), idarubicin (Idamycin) and epirubicin (Ellence), anthracyclin analogs such as mitoxantrone, actinimycin D, non-intercalating topoisomerase inhibitors such as epipodophyllotoxins (etoposide or VP16, teniposide or VM-26), podophylotoxin, bleomycin (Bleo), pepleomycin, compounds that form adducts with nucleic acid including platinum derivatives, e.g., cisplatin (CDDP), trans analog of cisplatin, carboplatin, iproplatin, tetraplatin and oxaliplatin, as well as camptothecin, topotecan, irinotecan (CPT-11), and SN-38. Specific examples of nucleic acid damaging treatments include radiation e.g., ultraviolet (UV), infrared (IR), or α-, β-, or γ-radiation, as well as environmental shock, e.g., hyperthermia. One of skill in the art can identify and use other DNA-damaging agents and treatments.

### D. CHEMICAL DEFINITIONS

As used herein, a “small molecule” refers to an organic compound that is either synthesized via conventional organic chemistry methods (e.g., in a laboratory) or found in nature. Typically, a small molecule is characterized in that it contains several carbon-carbon bonds, and has a molecular weight of less than about 1500 grams/mole. In certain embodiments, small molecules are less than about 1000 grams/mole. In certain embodiments, small molecules are less than about 550 grams/mole. In certain embodiments, small molecules are between about 200 and about 550 grams/mole. In certain embodiments, small molecules exclude peptides (e.g., compounds comprising 2 or more amino acids joined by a peptidyl bond). In certain embodiments, small molecules exclude nucleic acids.

As used herein, the term “amino” means —NH₂; the term “nitro” means —NO₂; the term “halo” designates —F, —Cl, —Br or —I; the term “mercapto” means —SH; the term “cyano” means —CN; the term “azido” means —N3; the term “silyl” means —SiH₃, and the term “hydroxy” means —OH.

As used herein, a “monovalent anion” refers to anions of a −1 charge. Such anions are well-known to those of skill in the art. Non-limiting examples of monovalent anions include halides (e.g., F—, Cl—, Br— and I—), NO₂—, NO₃—, hydroxide (OH—) and azide (N₃—).

As used herein, the structure indicates that the bond may be a single bond or a double bond. Those of skill in the chemical arts understand that in certain circumstances, a double bond between two particular atoms is chemically feasible and in certain circumstances, a double bond is not. The present invention therefore contemplates that a double bond may be formed only when chemically feasible.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cₙ-alkyl, and heteroatom-substituted Cₙ-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cₙ-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cₙ-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CHF, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cₙ-alkenyl, and heteroatom-substituted Cₙ-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cₙ-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “heteroatom-substituted Cₙ-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, —CH═CHF, —CH═CHCl and —CH═CHBr, are non-limiting examples of heteroatom-substituted alkenyl groups.

The term “aryl” includes heteroatom-unsubstituted aryl, heteroatom-substituted aryl, heteroatom-unsubstituted Cₙ-aryl, heteroatom-substituted Cₙ-aryl, heteroaryl, heterocyclic aryl groups, carbocyclic aryl groups, biaryl groups, and single-valent radicals derived from polycyclic fused hydrocarbons (PAHs). The term “heteroatom-unsubstituted Cₙ-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH═CHCH₃, —C₆H₄C≡CH, —C₆H₄C≡CCH₃, naphthyl, and the radical derived from biphenyl. The term “heteroatom-substituted Cₙ-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl.

The term “aralkyl” includes heteroatom-unsubstituted aralkyl, heteroatom-substituted aralkyl, heteroatom-unsubstituted Cₙ-aralkyl, heteroatom-substituted Cₙ-aralkyl, heteroaralkyl, and heterocyclic aralkyl groups. In certain embodiments, lower aralkyls are contemplated. The term “lower aralkyl” refers to aralkyls of 7-12 carbon atoms (that is, 7, 8, 9, 10, 11 or 12 carbon atoms). The term “heteroatom-unsubstituted Cₙ-aralkyl” refers to a radical, having a single saturated carbon atom as the point of attachment, further having a total of n carbon atoms, wherein at least 6 of the carbon atoms form an aromatic ring structure containing only carbon atoms, 7 or more hydrogen atoms, and no heteroatoms. For example, a heteroatom-unsubstituted C₇-C₁₀-aralkyl has 7 to 10 carbon atoms. Non-limiting examples of heteroatom-unsubstituted aralkyls are: phenylmethyl (benzyl, Bn) and phenylethyl. The term “heteroatom-substituted Cₙ-aralkyl” refers to a radical, having a single saturated carbon atom as the point of attachment, further having a total of n carbon atoms, 0, 1, or more than one hydrogen atom, and at least one heteroatom, wherein at least one of the carbon atoms is incorporated an aromatic ring structures, further wherein each heteroatom is independently selected from the group consisting of N, O, F, Cl, Br, I, Si, P, and S. For example, a heteroatom-substituted C₂-C₁₀-heteroaralkyl has 2 to 10 carbon atoms.

The term “acyl” includes straight-chain acyl, branched-chain acyl, cycloacyl, cyclic acyl, heteroatom-unsubstituted acyl, heteroatom-substituted acyl, heteroatom-unsubstituted Cₙ-acyl, heteroatom-substituted Cₙ-acyl, alkylcarbonyl, alkoxycarbonyl and aminocarbonyl groups. In certain embodiments, lower acyls are contemplated. The term “lower acyl” refers to acyls of 1-6 carbon atoms (that is, 1, 2, 3, 4, 5 or 6 carbon atoms). The term “heteroatom-unsubstituted Cₙ-acyl” refers to a radical, having a single carbon atom of a carbonyl group as the point of attachment, further having a linear or branched, cyclic or acyclic structure, further having a total of n carbon atoms, 1 or more hydrogen atoms, a total of one oxygen atom, and no additional heteroatoms. For example, a heteroatom-unsubstituted C₁-C₁₀-acyl has 1 to 10 carbon atoms. The groups, —CHO, —C(O)CH₃, —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, and —COC₆H₃(CH₃)₂, are non-limiting examples of heteroatom-unsubstituted acyl groups. The term “heteroatom-substituted Cₙ-acyl” refers to a radical, having a single carbon atom as the point of attachment, the carbon atom being part of a carbonyl group, further having a linear or branched, cyclic or acyclic structure, further having a total of n carbon atoms, 0, 1, or more than one hydrogen atom, at least one additional heteroatom, in addition to the oxygen of the carbonyl group, wherein each additional heteroatom is independently selected from the group consisting of N, O, F, Cl, Br, I, Si, P, and S. For example, a heteroatom-substituted C₁-C₁₀-acyl has 1 to 10 carbon atoms. The groups, —C(O)CH₂CF₃, —CO₂H, CO₂, —CO₂CH₃, —CO₂CH₂CH₃, —CO₂CH₂CH₂CH₃, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, CONHCH(CH₂)₂, —CON(CH₃)₂, and —CONHCH₂CF₃, are non-limiting examples of heteroatom-substituted acyl groups.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cₙ-alkoxy, and heteroatom-substituted Cₙ-alkoxy. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cₙ-alkoxy” refers to a group, having the structure —OR, in which R is a heteroatom-unsubstituted Cₙ-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cₙ-alkoxy” refers to a group, having the structure —OR, in which R is a heteroatom-substituted Cₙ-alkyl, as that term is defined above. For example, —OCH₂CF₃ is a heteroatom-substituted alkoxy group.

The term “alkenyloxy” includes straight-chain alkenyloxy, branched-chain alkenyloxy, cycloalkenyloxy, cyclic alkenyloxy, heteroatom-unsubstituted alkenyloxy, heteroatom-substituted alkenyloxy, heteroatom-unsubstituted Cₙ-alkenyloxy, and heteroatom-substituted Cₙ-alkenyloxy. The term “heteroatom-unsubstituted Cₙ-alkenyloxy” refers to a group, having the structure —OR, in which R is a heteroatom-unsubstituted Cₙ-alkenyl, as that term is defined above. The term “heteroatom-substituted Cₙ-alkenyloxy” refers to a group, having the structure —OR, in which R is a heteroatom-substituted Cₙ-alkenyl, as that term is defined above.

The term “alkynyloxy” includes straight-chain alkynyloxy, branched-chain alkynyloxy, cycloalkynyloxy, cyclic alkynyloxy, heteroatom-unsubstituted alkynyloxy, heteroatom-substituted alkynyloxy, heteroatom-unsubstituted Cₙ-alkynyloxy, and heteroatom-substituted Cₙ-alkynyloxy. The term “heteroatom-unsubstituted Cₙ-alkynyloxy” refers to a group, having the structure —OR, in which R is a heteroatom-unsubstituted Cₙ-alkynyl, as that term is defined above. The term “heteroatom-substituted Cₙ-alkynyloxy” refers to a group, having the structure —OR, in which R is a heteroatom-substituted Cₙ-alkynyl, as that term is defined above.

The term “aryloxy” includes heteroatom-unsubstituted aryloxy, heteroatom-substituted aryloxy, heteroatom-unsubstituted Cₙ-aryloxy, heteroatom-substituted Cₙ-aryloxy, heteroaryloxy, and heterocyclic aryloxy groups. The term “heteroatom-unsubstituted Cₙ-aryloxy” refers to a group, having the structure —OAr, in which Ar is a heteroatom-unsubstituted Cₙ-aryl, as that term is defined above. A non-limiting example of a heteroatom-unsubstituted aryloxy group is —OC₆H₅. The term “heteroatom-substituted Cₙ-aryloxy” refers to a group, having the structure —OAr, in which Ar is a heteroatom-substituted Cₙ-aryl, as that term is defined above.

The term “aralkyloxy” includes heteroatom-unsubstituted aralkyloxy, heteroatom-substituted aralkyloxy, heteroatom-unsubstituted Cₙ-aralkyloxy, heteroatom-substituted Cₙ-aralkyloxy, heteroaralkyloxy, and heterocyclic aralkyloxy groups. The term “heteroatom-unsubstituted Cₙ-aralkyloxy” refers to a group, having the structure —OAr, in which Ar is a heteroatom-unsubstituted Cₙ-aralkyl, as that term is defined above. The term “heteroatom-substituted Cₙ-aralkyloxy” refers to a group, having the structure —OAr, in which Ar is a heteroatom-substituted Cₙ-aralkyl, as that term is defined above.

The term “acyloxy” includes straight-chain acyloxy, branched-chain acyloxy, cycloacyloxy, cyclic acyloxy, heteroatom-unsubstituted acyloxy, heteroatom-substituted acyloxy, heteroatom-unsubstituted Cₙ-acyloxy, heteroatom-substituted Cₙ-acyloxy, alkylcarbonyloxy, arylcarbonyloxy, alkoxycarbonyloxy, aryloxycarbonyloxy, and carboxylate groups. The term “heteroatom-unsubstituted Cₙ-acyloxy” refers to a group, having the structure —OAc, in which Ac is a heteroatom-unsubstituted Cₙ-acyl, as that term is defined above. For example, —OC(O)CH₃ is a non-limiting example of a heteroatom-unsubstituted acyloxy group. The term “heteroatom-substituted Cₙ-acyloxy” refers to a group, having the structure —OAc, in which Ac is a heteroatom-substituted Cₙ-acyl, as that term is defined above. For example, —OC(O)OCH₃ and —OC(O)NHCH₃ are non-limiting examples of heteroatom-unsubstituted acyloxy groups.

The term “alkylamino” includes straight-chain alkylamino, branched-chain alkylamino, cycloalkylamino, cyclic alkylamino, heteroatom-unsubstituted alkylamino, heteroatom-substituted alkylamino, heteroatom-unsubstituted Cₙ-alkylamino, and heteroatom-substituted Cₙ-alkylamino. The term “heteroatom-unsubstituted Cₙ-alkylamino” refers to a radical, having a single nitrogen atom as the point of attachment, further having one or two saturated carbon atoms attached to the nitrogen atom, further having a linear or branched, cyclic or acyclic structure, containing a total of n carbon atoms, all of which are nonaromatic, 4 or more hydrogen atoms, a total of 1 nitrogen atom, and no additional heteroatoms. For example, a heteroatom-unsubstituted C₁-C₁₀-alkylamino has 1 to 10 carbon atoms. The term “heteroatom-unsubstituted Cₙ-alkylamino” includes groups, having the structure —NHR, in which R is a heteroatom-unsubstituted Cₙ-alkyl, as that term is defined above. A heteroatom-unsubstituted alkylamino group would include —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —N(CH₃)₂, —N(CH₃)CH₂CH₃, —N(CH₂CH₃)₂, N-pyrrolidinyl, and N-piperidinyl. The term “heteroatom-substituted Cₙ-alkylamino” refers to a radical, having a single nitrogen atom as the point of attachment, further having one or two saturated carbon atoms attached to the nitrogen atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₁-C₁₀-alkylamino has 1 to 10 carbon atoms. The term “heteroatom-substituted Cₙ-alkylamino” includes groups, having the structure —NHR, in which R is a heteroatom-substituted Cₙ-alkyl, as that term is defined above.

The term “alkenylamino” includes straight-chain alkenylamino, branched-chain alkenylamino, cycloalkenylamino, cyclic alkenylamino, heteroatom-unsubstituted alkenylamino, heteroatom-substituted alkenylamino, heteroatom-unsubstituted Cₙ-alkenylamino, heteroatom-substituted Cₙ-alkenylamino, dialkenylamino, and alkyl(alkenyl)amino groups. The term “heteroatom-unsubstituted Cₙ-alkenylamino” refers to a radical, having a single nitrogen atom as the point of attachment, further having one or two carbon atoms attached to the nitrogen atom, further having a linear or branched, cyclic or acyclic structure, containing at least one nonaromatic carbon-carbon double bond, a total of n carbon atoms, 4 or more hydrogen atoms, a total of one nitrogen atom, and no additional heteroatoms. For example, a heteroatom-unsubstituted C₂-C₁₀-alkenylamino has 2 to 10 carbon atoms. The term “heteroatom-unsubstituted Cₙ-alkenylamino” includes groups, having the structure —NHR, in which R is a heteroatom-unsubstituted Cₙ-alkenyl, as that term is defined above. The term “heteroatom-substituted Cₙ-alkenylamino” refers to a radical, having a single nitrogen atom as the point of attachment and at least one nonaromatic carbon-carbon double bond, but no carbon-carbon triple bonds, further having one or two carbon atoms attached to the nitrogen atom, further having a linear or branched, cyclic or acyclic structure, further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkenylamino has 2 to 10 carbon atoms. The term “heteroatom-substituted Cₙ-alkenylamino” includes groups, having the structure —NHR, in which R is a heteroatom-substituted Cₙ-alkenyl, as that term is defined above.

The term “alkynylamino” includes straight-chain alkynylamino, branched-chain alkynylamino, cycloalkynylamino, cyclic alkynylamino, heteroatom-unsubstituted alkynylamino, heteroatom-substituted alkynylamino, heteroatom-unsubstituted Cₙ-alkynylamino, heteroatom-substituted Cₙ-alkynylamino, dialkynylamino, alkyl(alkynyl)amino, and alkenyl(alkynyl)amino groups. The term “heteroatom-unsubstituted Cₙ-alkynylamino” refers to a radical, having a single nitrogen atom as the point of attachment, further having one or two carbon atoms attached to the nitrogen atom, further having a linear or branched, cyclic or acyclic structure, containing at least one carbon-carbon triple bond, a total of n carbon atoms, at least one hydrogen atoms, a total of one nitrogen atom, and no additional heteroatoms. For example, a heteroatom-unsubstituted C₂-C₁₀-alkynylamino has 2 to 10 carbon atoms. The term “heteroatom-unsubstituted Cₙ-alkynylamino” includes groups, having the structure —NHR, in which R is a heteroatom-unsubstituted Cₙ-alkynyl, as that term is defined above. The term “heteroatom-substituted Cₙ-alkynylamino” refers to a radical, having a single nitrogen atom as the point of attachment, further having one or two carbon atoms attached to the nitrogen atom, further having at least one nonaromatic carbon-carbon triple bond, further having a linear or branched, cyclic or acyclic structure, and further having a total of n carbon atoms, 0, 1, or more than one hydrogen atom, and at least one additional heteroatom, that is, in addition to the nitrogen atom at the point of attachment, wherein each additional heteroatom is independently selected from the group consisting of N, O, F, Cl, Br, I, Si, P, and S. For example, a heteroatom-substituted C₂-C₁₀-alkynylamino has 2 to 10 carbon atoms. The term “heteroatom-substituted Cₙ-alkynylamino” includes groups, having the structure —NHR, in which R is a heteroatom-substituted Cₙ-alkynyl, as that term is defined above.

The term “arylamino” includes heteroatom-unsubstituted arylamino, heteroatom-substituted arylamino, heteroatom-unsubstituted Cₙ-arylamino, heteroatom-substituted Cₙ-arylamino, heteroarylamino, heterocyclic arylamino, and alkyl(aryl)amino groups. The term “heteroatom-unsubstituted Cₙ-arylamino” refers to a radical, having a single nitrogen atom as the point of attachment, further having at least one aromatic ring structure attached to the nitrogen atom, wherein the aromatic ring structure contains only carbon atoms, further having a total of n carbon atoms, 6 or more hydrogen atoms, a total of one nitrogen atom, and no additional heteroatoms. For example, a heteroatom-unsubstituted C₆-C₁₀-arylamino has 6 to 10 carbon atoms. The term “heteroatom-unsubstituted Cₙ-arylamino” includes groups, having the structure —NHR, in which R is a heteroatom-unsubstituted Cₙ-aryl, as that term is defined above. The term “heteroatom-substituted Cₙ-arylamino” refers to a radical, having a single nitrogen atom as the point of attachment, further having a total of n carbon atoms, at least one hydrogen atom, at least one additional heteroatoms, that is, in addition to the nitrogen atom at the point of attachment, wherein at least one of the carbon atoms is incorporated into one or more aromatic ring structures, further wherein each additional heteroatom is independently selected from the group consisting of N, O, F, Cl, Br, I, Si, P, and S. For example, a heteroatom-substituted C₆-C₁₀-arylamino has 6 to 10 carbon atoms. The term “heteroatom-substituted Cₙ-arylamino” includes groups, having the structure —NHR, in which R is a heteroatom-substituted Cₙ-aryl, as that term is defined above.

The term “aralkylamino” includes heteroatom-unsubstituted aralkylamino, heteroatom-substituted aralkylamino, heteroatom-unsubstituted Cₙ-aralkylamino, heteroatom-substituted Cₙ-aralkylamino, heteroaralkylamino, heterocyclic aralkylamino groups, and diaralkylamino groups. The term “heteroatom-unsubstituted Cₙ-aralkylamino” refers to a radical, having a single nitrogen atom as the point of attachment, further having one or two saturated carbon atoms attached to the nitrogen atom, further having a total of n carbon atoms, wherein at least 6 of the carbon atoms form an aromatic ring structure containing only carbon atoms, 8 or more hydrogen atoms, a total of one nitrogen atom, and no additional heteroatoms. For example, a heteroatom-unsubstituted C₇-C₁₀-aralkylamino has 7 to 10 carbon atoms. The term “heteroatom-unsubstituted Cₙ-aralkylamino” includes groups, having the structure —NHR, in which R is a heteroatom-unsubstituted Cₙ-aralkyl, as that term is defined above. The term “heteroatom-substituted Cₙ-aralkylamino” refers to a radical, having a single nitrogen atom as the point of attachment, further having at least one or two saturated carbon atoms attached to the nitrogen atom, further having a total of n carbon atoms, 0, 1, or more than one hydrogen atom, at least one additional heteroatom, that is, in addition to the nitrogen atom at the point of attachment, wherein at least one of the carbon atom incorporated into an aromatic ring, further wherein each heteroatom is independently selected from the group consisting of N, O, F, Cl, Br, I, Si, P, and S. For example, a heteroatom-substituted C₇-C₁₀-aralkylamino has 7 to 10 carbon atoms. The term “heteroatom-substituted Cₙ-aralkylamino” includes groups, having the structure —NHR, in which R is a heteroatom-substituted Cₙ-aralkyl, as that term is defined above.

The term “amido” includes straight-chain amido, branched-chain amido, cycloamido, cyclic amido, heteroatom-unsubstituted amido, heteroatom-substituted amido, heteroatom-unsubstituted Cₙ-amido, heteroatom-substituted Cₙ-amido, alkylcarbonylamino, arylcarbonylamino, alkoxycarbonylamino, aryloxycarbonylamino, acylamino, alkylaminocarbonylamino, arylaminocarbonylamino, and ureido groups. The term “heteroatom-unsubstituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n carbon atoms, 1 or more hydrogen atoms, a total of one oxygen atom, a total of one nitrogen atom, and no additional heteroatoms. For example, a heteroatom-unsubstituted C₁-C₁₀-amido has 1 to 10 carbon atoms. The term “heteroatom-unsubstituted Cₙ-amido” includes groups, having the structure —NHR, in which R is a heteroatom-unsubstituted Cₙ-acyl, as that term is defined above. The group, —NHC(O)CH₃, is a non-limiting example of a heteroatom-unsubstituted amido group. The term “heteroatom-substituted Cₙ-amido” refers to a radical, having a single nitrogen atom as the point of attachment, further having a carbonyl group attached via its carbon atom to the nitrogen atom, further having a linear or branched, cyclic or acyclic structure, further having a total of n aromatic or nonaromatic carbon atoms, 0, 1, or more than one hydrogen atom, at least one additional heteroatom in addition to the oxygen of the carbonyl group, wherein each additional heteroatom is independently selected from the group consisting of N, O, F, Cl, Br, I, Si, P, and S. For example, a heteroatom-substituted C₁-C₁₀-amido has 1 to 10 carbon atoms. The term “heteroatom-substituted Cₙ-amido” includes groups, having the structure —NHR, in which R is a heteroatom-unsubstituted Cₙ-acyl, as that term is defined above. The group, —NHCO₂CH₃, is a non-limiting example of a heteroatom-substituted amido group.

The term “alkylthio” includes straight-chain alkylthio, branched-chain alkylthio, cycloalkylthio, cyclic alkylthio, heteroatom-unsubstituted alkylthio, heteroatom-substituted alkylthio, heteroatom-unsubstituted Cₙ-alkylthio, and heteroatom-substituted Cₙ-alkylthio. The term “heteroatom-unsubstituted Cₙ-alkylthio” refers to a group, having the structure —SR, in which R is a heteroatom-unsubstituted Cₙ-alkyl, as that term is defined above. The group, —SCH₃, is an example of a heteroatom-unsubstituted alkylthio group. The term “heteroatom-substituted Cₙ-alkylthio” refers to a group, having the structure —SR, in which R is a heteroatom-substituted Cₙ-alkyl, as that term is defined above.

The term “alkenylthio” includes straight-chain alkenylthio, branched-chain alkenylthio, cycloalkenylthio, cyclic alkenylthio, heteroatom-unsubstituted alkenylthio, heteroatom-substituted alkenylthio, heteroatom-unsubstituted Cₙ-alkenylthio, and heteroatom-substituted Cₙ-alkenylthio. The term “heteroatom-unsubstituted Cₙ-alkenylthio” refers to a group, having the structure —SR, in which R is a heteroatom-unsubstituted Cₙ-alkenyl, as that term is defined above. The term “heteroatom-substituted Cₙ-alkenylthio” refers to a group, having the structure —SR, in which R is a heteroatom-substituted Cₙ-alkenyl, as that term is defined above.

The term “alkynylthio” includes straight-chain alkynylthio, branched-chain alkynylthio, cycloalkynylthio, cyclic alkynylthio, heteroatom-unsubstituted alkynylthio, heteroatom-substituted alkynylthio, heteroatom-unsubstituted Cₙ-alkynylthio, and heteroatom-substituted Cₙ-alkynylthio. The term “heteroatom-unsubstituted Cₙ-alkynylthio” refers to a group, having the structure —SR, in which R is a heteroatom-unsubstituted Cₙ-alkynyl, as that term is defined above. The term “heteroatom-substituted Cₙ-alkynylthio” refers to a group, having the structure —SR, in which R is a heteroatom-substituted Cₙ-alkynyl, as that term is defined above.

The term “arylthio” includes heteroatom-unsubstituted arylthio, heteroatom-substituted arylthio, heteroatom-unsubstituted Cₙ-arylthio, heteroatom-substituted Cₙ-arylthio, heteroarylthio, and heterocyclic arylthio groups. The term “heteroatom-unsubstituted Cₙ-arylthio” refers to a group, having the structure —SAr, in which Ar is a heteroatom-unsubstituted Cₙ-aryl, as that term is defined above. The group, —SC₆H₅, is an example of a heteroatom-unsubstituted arylthio group. The term “heteroatom-substituted Cₙ-arylthio” refers to a group, having the structure —SAr, in which Ar is a heteroatom-substituted Cₙ-aryl, as that term is defined above.

The term “aralkylthio” includes heteroatom-unsubstituted aralkylthio, heteroatom-substituted aralkylthio, heteroatom-unsubstituted Cₙ-aralkylthio, heteroatom-substituted Cₙ-aralkylthio, heteroaralkylthio, and heterocyclic aralkylthio groups. The term “heteroatom-unsubstituted Cₙ-aralkylthio” refers to a group, having the structure —SAr, in which Ar is a heteroatom-unsubstituted Cₙ-aralkyl, as that term is defined above. The group, —SCH₂C₆H₅, is an example of a heteroatom-unsubstituted aralkyl group. The term “heteroatom-substituted Cₙ-aralkylthio” refers to a group, having the structure —SAr, in which Ar is a heteroatom-substituted Cₙ-aralkyl, as that term is defined above.

The term “acylthio” includes straight-chain acylthio, branched-chain acylthio, cycloacylthio, cyclic acylthio, heteroatom-unsubstituted acylthio, heteroatom-substituted acylthio, heteroatom-unsubstituted Cₙ-acylthio, heteroatom-substituted Cₙ-acylthio, alkylcarbonyloxy, arylcarbonyloxy, alkoxycarbonyloxy, aryloxycarbonyloxy, and carboxylate groups. The term “heteroatom-unsubstituted Cₙ-acylthio” refers to a group, having the structure —SAc, in which Ac is a heteroatom-unsubstituted Cₙ-acyl, as that term is defined above. The group, —SCOCH₃, is an example of a heteroatom-unsubstituted acylthio group. The term “heteroatom-substituted Cₙ-acylthio” refers to a group, having the structure —SAc, in which Ac is a heteroatom-substituted Cₙ-acyl, as that term is defined above.

The term “alkylsilyl” includes straight-chain alkylsilyl, branched-chain alkylsilyl, cycloalkylsilyl, cyclic alkylsilyl, heteroatom-unsubstituted alkylsilyl, heteroatom-substituted alkylsilyl, heteroatom-unsubstituted Cₙ-alkylsilyl, and heteroatom-substituted Cₙ-alkylsilyl. The term “heteroatom-unsubstituted Cₙ-alkylsilyl” refers to a radical, having a single silicon atom as the point of attachment, further having one, two, or three saturated carbon atoms attached to the silicon atom, further having a linear or branched, cyclic or acyclic structure, containing a total of n carbon atoms, all of which are nonaromatic, 5 or more hydrogen atoms, a total of 1 silicon atom, and no additional heteroatoms. For example, a heteroatom-unsubstituted C₁-C₁₀-alkylsilyl has 1 to 10 carbon atoms. An alkylsilyl group includes dialkylamino groups. The groups, —Si(CH₃)₃ and —Si(CH₃)₂C(CH₃)₃, are non-limiting examples of heteroatom-unsubstituted alkylsilyl groups. The term “heteroatom-substituted Cₙ-alkylsilyl” refers to a radical, having a single silicon atom as the point of attachment, further having at least one, two, or three saturated carbon atoms attached to the silicon atom, no carbon-carbon double or triple bonds, further having a linear or branched, cyclic or acyclic structure, further having a total of n carbon atoms, all of which are nonaromatic, 0, 1, or more than one hydrogen atom, and at least one additional heteroatom, that is, in addition to the silicon atom at the point of attachment, wherein each additional heteroatom is independently selected from the group consisting of N, O, F, Cl, Br, I, Si, P, and S. For example, a heteroatom-substituted C₁-C₁₀-alkylsilyl has 1 to 10 carbon atoms.

The term “phosphonate” includes straight-chain phosphonate, branched-chain phosphonate, cyclophosphonate, cyclic phosphonate, heteroatom-unsubstituted phosphonate, heteroatom-substituted phosphonate, heteroatom-unsubstituted Cₙ-phosphonate, and heteroatom-substituted Cₙ-phosphonate. The term “heteroatom-unsubstituted Cₙ-phosphonate” refers to a radical, having a single phosphorous atom as the point of attachment, further having a linear or branched, cyclic or acyclic structure, further having a total of n carbon atoms, 2 or more hydrogen atoms, a total of three oxygen atom, and no additional heteroatoms. The three oxygen atoms are directly attached to the phosphorous atom, with one of these oxygen atoms doubly bonded to the phosphorous atom. For example, a heteroatom-unsubstituted C₀-C₁₀-phosphonate has 0 to 10 carbon atoms. The groups, —P(O)(OH)₂, —P(O)(OH)OCH₃, —P(O)(OH)OCH₂CH₃, —P(O)(OCH₃)₂, and —P(O)(OH)(OC₆H₅) are non-limiting examples of heteroatom-unsubstituted phosphonate groups. The term “heteroatom-substituted Cₙ-phosphonate” refers to a radical, having a single phosphorous atom as the point of attachment, further having a linear or branched, cyclic or acyclic structure, further having a total of n carbon atoms, 2 or more hydrogen atoms, three or more oxygen atoms, three of which are directly attached to the phosphorous atom, with one of these three oxygen atoms doubly bonded to the phosphorous atom, and further having at least one additional heteroatom in addition to the three oxygen atoms, wherein each additional heteroatom is independently selected from the group consisting of N, O, F, Cl, Br, I, Si, P, and S. For example, a heteroatom-unsubstituted C₀-C₁₀-phosphonate has 0 to 10 carbon atoms.

The term “phosphinate” includes straight-chain phosphinate, branched-chain phosphinate, cyclophosphinate, cyclic phosphinate, heteroatom-unsubstituted phosphinate, heteroatom-substituted phosphinate, heteroatom-unsubstituted Cₙ-phosphinate, and heteroatom-substituted Cₙ-phosphinate. The term “heteroatom-unsubstituted Cₙ-phosphinate” refers to a radical, having a single phosphorous atom as the point of attachment, further having a linear or branched, cyclic or acyclic structure, further having a total of n carbon atoms, 2 or more hydrogen atoms, a total of two oxygen atom, and no additional heteroatoms. The two oxygen atoms are directly attached to the phosphorous atom, with one of these oxygen atoms doubly bonded to the phosphorous atom. For example, a heteroatom-unsubstituted C₀-C₁₀-phosphinate has 0 to 10 carbon atoms. The groups, —P(O)(OH)H, —P(O)(OH)CH₃, —P(O)(OH)CH₂CH₃, —P(O)(OCH₃)CH₃, and —P(O)(OC₆H₅)H are non-limiting examples of heteroatom-unsubstituted phosphinate groups. The term “heteroatom-substituted Cₙ-phosphinate” refers to a radical, having a single phosphorous atom as the point of attachment, further having a linear or branched, cyclic or acyclic structure, further having a total of n carbon atoms, 2 or more hydrogen atoms, two or more oxygen atoms, two of which are directly attached to the phosphorous atom, with one of these two oxygen atoms doubly bonded to the phosphorous atom, and further having at least one additional heteroatom in addition to the two oxygen atoms, wherein each additional heteroatom is independently selected from the group consisting of N, O, F, Cl, Br, I, Si, P, and S. For example, a heteroatom-unsubstituted C₀-C₁₀-phosphinate has 0 to 10 carbon atoms.

Compounds described herein may be prepared synthetically using conventional organic chemistry methods known to those of skill in the art and/or are commercially available (e.g., ChemBridge Co., San Diego, Calif.).

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Derivatives of compounds of the present invention are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (2002), which is incorporated herein by reference.

### E. PHARMACEUTICAL FORMULATIONS AND ADMINISTRATION THEREOF

1. Pharmaceutical Formulations and Routes of Administration

Pharmaceutical compositions of the present invention comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, systemically, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a compound of the present invention. In other embodiments, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin, or combinations thereof.

2. Combination Therapy

In some embodiments, it is contemplated that the RAD51 stimulators of the invention may be used in conjunction with additional therapeutic agents as part of a treatment regimen. This process may involve contacting cell(s) or administering to the subject the agents at the same time or within a period of time wherein separate administration of the agents produces a desired therapeutic benefit. This may be achieved by contacting the cell, tissue or organism with a single composition or pharmacological formulation that includes two or more agents, or by contacting the cell with two or more distinct compositions or formulations, wherein one composition includes one agent and the other includes another.

The compounds of the present invention may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the RAD51 stimulator. In other aspects, one or more additional agents may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering the RAD51 modulator.

Various combination regimens of the agents may be employed. Non-limiting examples of such combinations are shown below, wherein a RAD51 stimulator is “A” and a second agent is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

In some embodiments, more than one course of therapy may be employed. It is contemplated that multiple courses may be implemented. In certain embodiments, a patient may have previously undergone radiation or chemotherapy for a cancer that turns out to be chemotherapy- or radiation-resistant. Alternatively, a patient may have a recurring cancer.

In some embodiments, it is contemplated that RAD51 stimulators may be used as a therapy alone and not in combination with any other therapeutic agent. In particular it is contemplated that RAD51 stimulators may be used without any additional DNA damaging agent.

### F. ORGANISMS AND CELL SOURCE

Cells that may be used in many methods of the invention can be from a variety of sources. Embodiments include the use of mammalian cells, such as cells from monkeys, chimpanzees, rabbits, mice, rats, ferrets, dogs, pigs, humans, and cows. Alternatively, the cells may be from fruit flies, yeast, or E. Coli, which are all model systems for evaluating homologous recombination.

Methods of the invention can involve cells, tissues, or organs involving the heart, lung, kidney, liver, bone marrow, pancreas, skin, bone, vein, artery, cornea, blood, small intestine, large intestine, brain, spinal cord, smooth muscle, skeletal muscle, ovary, testis, uterus, and umbilical cord.

Moreover, methods can be employed in cells of the following type: platelet, myelocyte, erythrocyte, lymphocyte, adipocyte, fibroblast, epithelial cell, endothelial cell, smooth muscle cell, skeletal muscle cell, endocrine cell, glial cell, neuron, secretory cell, barrier function cell, contractile cell, absorptive cell, mucosal cell, limbus cell (from cornea), stem cell (totipotent, pluripotent or multipotent), unfertilized or fertilized oocyte, or sperm.

Moreover, methods can be implemented with or in plants or parts of plants, including fruit, flowers, leaves, stems, seeds, cuttings. Plants can be agricultural, medicinal, or decorative.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of RAD51 Stimulators

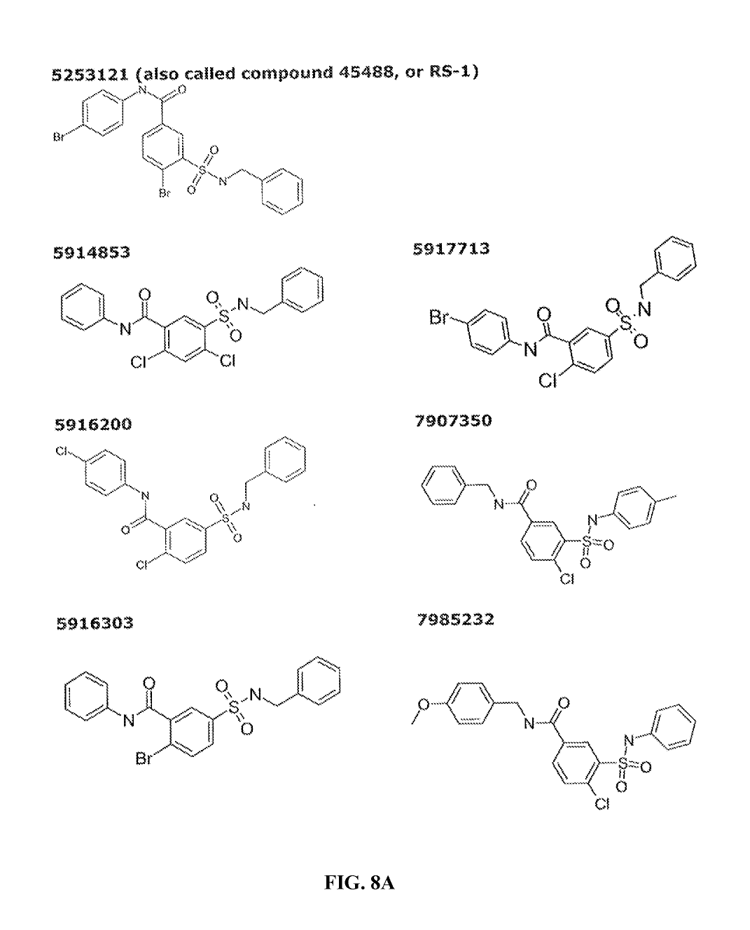

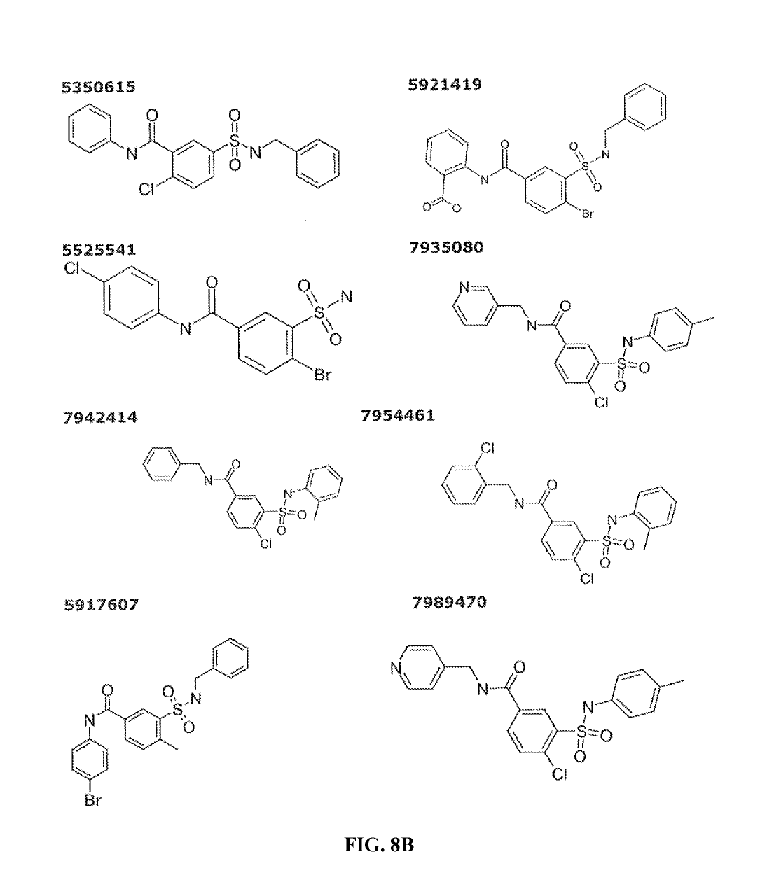

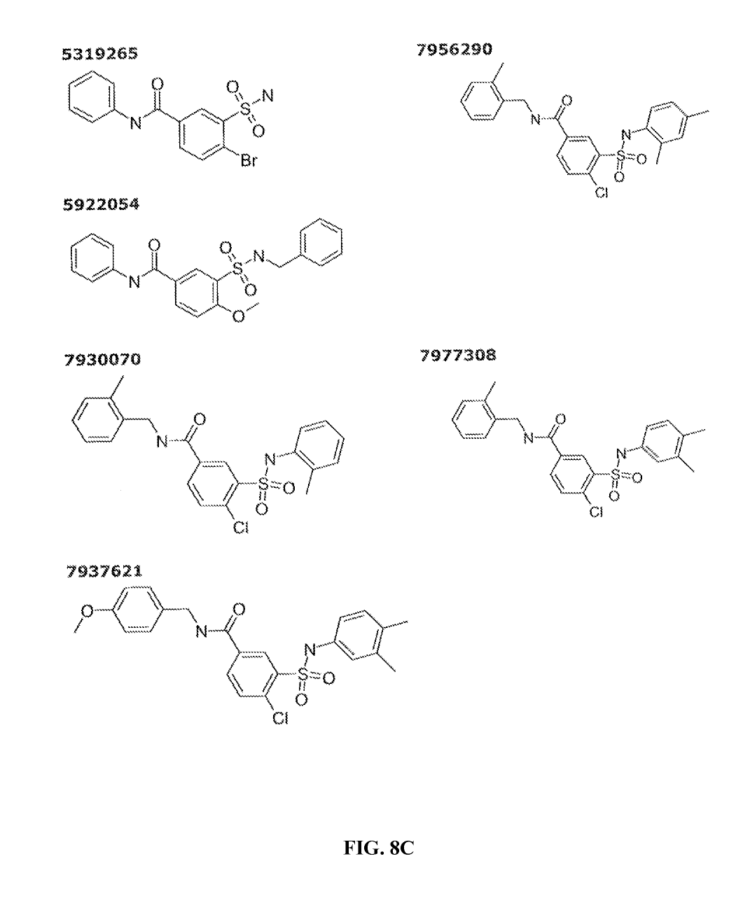

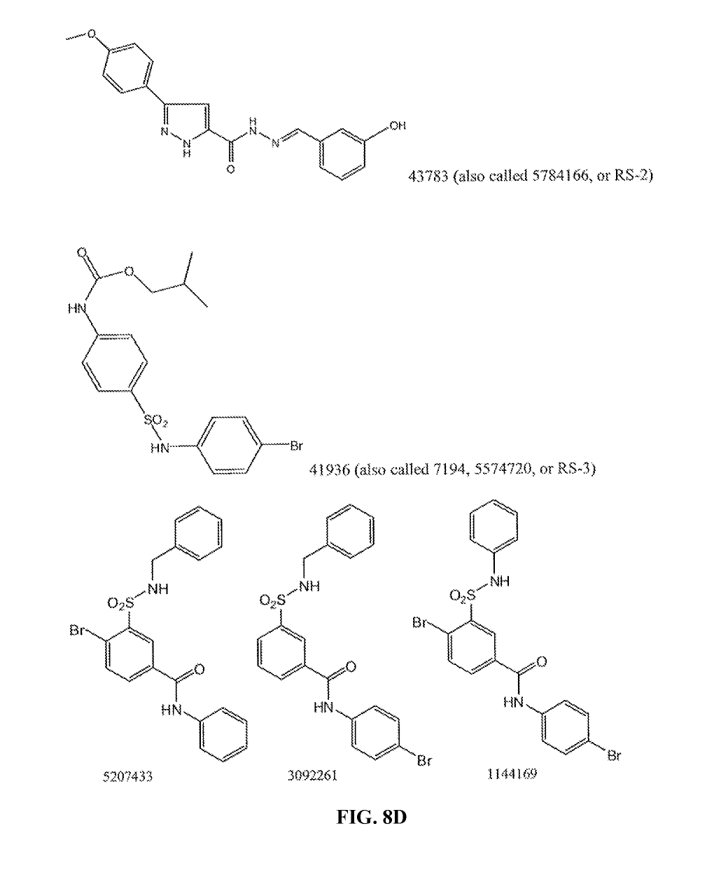

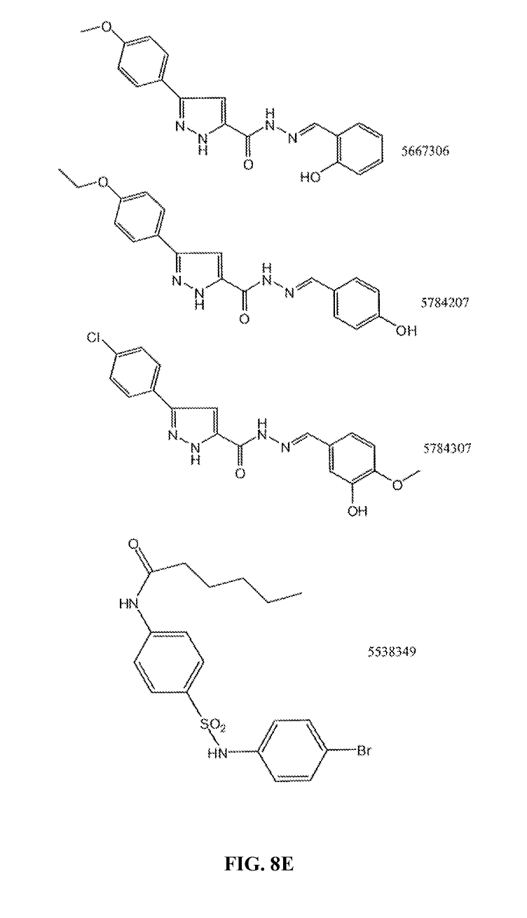

Small molecule RAD51 stimulators were identified from a screen of a small-molecule chemical library as disclosed in Connell et. al. (US 2010/0248371), which is hereby incorporated by reference in its entirety. Briefly, a fluorescence polarization assay for RAD51 filament formation was used to screen a 10,000 compound small-molecule library (Chembridge DIVERSet collection) for compounds that stimulate RAD51 filament formation. The screen identified three small molecule compounds that stimulate RAD51 filament formation by at least 50% (FIGS. 8A-8E, compounds 45488 (“RS-1”), 43783, and 41936). Further study of RS-1 confirmed that it enhances RAD51 filament formation and that it protects these filaments from buffers containing high salt concentration (which typically destabilize RAD51 filaments). Imaging with electron microscopy confirmed that the increases in measured fluorescence polarization were, in fact, due to compound-stimulated filaments with long track lengths.

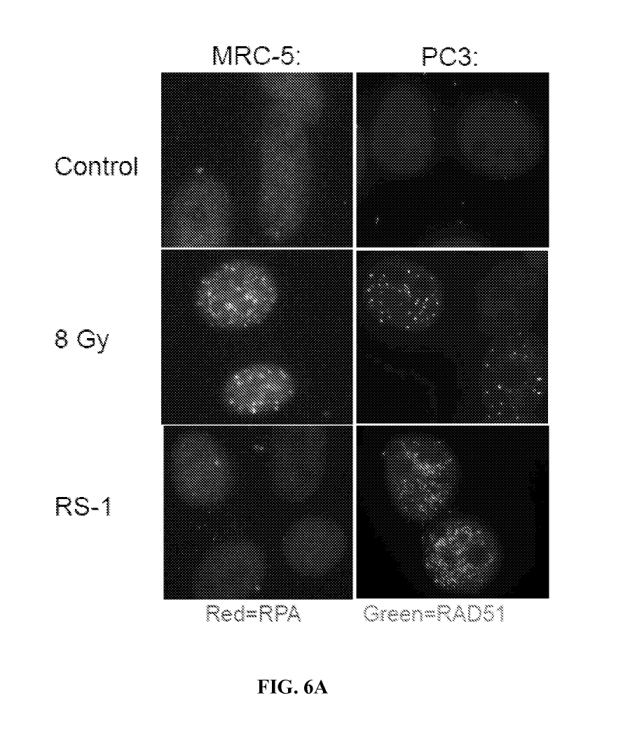

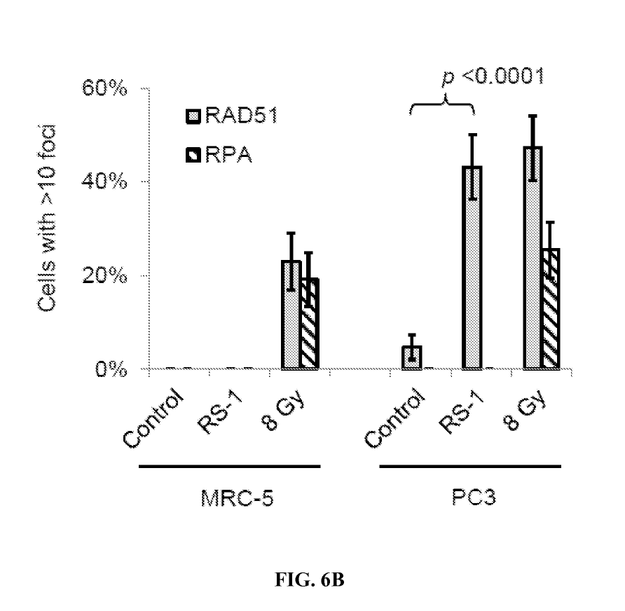

RS-1 was also tested using an assay that tests strand invasion, a later step in homologous recombination. In this assay a ³²P-labeled ssDNA oligonucleotide is incubated with a supercoiled double-stranded plasmid, which contains an area of homology to the ssDNA. RAD51 can catalyze the formation of a joint molecule which is detected as a unique band after electrophoresis (Wiese et al., 2002). These experiments demonstrated that RS-1 is capable of stimulating DNA strand invasion activity of RAD51 (FIG. 6B).

Additional compounds were identified in the Cambridge library that shared varying degrees of structural similarity to RS 1. These are also shown in FIGS. 8A-8E.

### Example 2

### Chemical Synthesis of Compounds

3-Benzylsulfamoyl-4-bromo-N-(4-bromo-phenyl)-benzamide was synthetized by reaction of chlorosulfonic acid with 4-bromobenzoic acid followed by sulfonamide formation with benzylamine and coupling with 4-bromoaniline. ¹H NMR and ¹³C NMR spectra were obtained using a Bruker spectrometer with TMS as an internal standard. The following abbreviations indicating multiplicity were used: s=singlet, d=doublet, t=triplet, m=multiplet. HRMS experiments were carried out using a Shimadzu IT-TOF instrument with MeCN and H₂O spiked with 0.1% formic acid as the mobile phase. Reaction progress was monitored by TLC using precoated silica gel plates (Merck silica gel 60 F254, 250 μm thickness). Preparative HPLC was carried out using a Shimadzu preparative liquid chromatograph with the following specifications: column, ACE 5 AQ (150 mm×21.2 mm) with 5 μm particle size; gradient, 25-100% MeOH/H₂O, 30 min; 100% MeOH, 5 min; 100-25% MeOH/H₂O, 4 min; 25% MeOH/H₂O, 1 min; flow rate=17 mL/min with wavelength monitoring at 254 and 280 nm. Both solvents were spiked with 0.05% TFA. Analytical HPLC was carried out using an Agilent 1100 series instrument with the following specifications: column, Luna 5 μm C18(2) 100 Å (150 mm×4.60 mm) with 5 μm particle size; flow rate=1.4 mL/min with wavelength monitoring at 254 nm; gradient, 10-100% MeOH/H₂O, 18 min; 100% MeOH, 3 min; 100-10% MeOH/H₂O, 3 min; 10% MeOH/H₂O, 5 min. Both solvents were spiked with 0.05% TFA. The purity of all tested compounds was >98%.

3-Benzylsulfamoyl-4-bromo-benzoic acid (JK-4-36): 4-bromobenzoic acid (1.7 g, 8.5 mmol) was added drop-wise to chlorosulfonic acid (4.3 mL, 8.5 mmol) at 0° C. After addition was finished reaction mixture was heated to 130° C. for 10 h. Cooled reaction mixture was added drop-wise to 85 mL of ice water. Formed precipitate was filtered off and washed with cold water. Solid was dissolved in diethyl ether and dried with sodium sulfate. Solvent was evapotarted giving 4-bromo-3-chlorosulfonyl-benzoic acid (2 g, 6.67 mmol, 79%) as beige solid. 4-bromo-3-chlorosulfonyl-benzoic acid was dissolved in 8 mL of THF. To this solution benzylamine (0.73 mL, 6.67 mmol) was added drop-wise and reaction was refluxed for 18 h. After that time 15 mL of ethyl acetate and 15 mL of 1 M NaOH were added. Organic phase was further extracted with two 20 mL portions of 1 M NaOH. Aqueous phases were combined, pH adjusted to approx. 2 with 1 M HCl and extracted with three 15 mL portions of ethyl acetate. Organic phases were dried with sodium sulfate. Evaporation of solvent gave 568 mg (23%) of 3-benzylsulfamoyl-4-bromo-benzoic acid as white solid. ¹H-NMR (400 MHz, DMSO-d₆): δ 8.55 (t, J=6.1 Hz, 1H), 8.40 (d, J=1.9 Hz, 1H), 7.92 (m, 2H), 7.16 (m, 5H), 4.14 (d, J=4.0 Hz, 2H). ¹³C-NMR (100 MHz, DMSO-d₆): δ 166.4, 140.7, 140.6, 137.7, 136.0, 134.2, 131.4, 128.5, 128.0, 127.5, 124.0, 46.6.

3-Benzylsulfamoyl-4-bromo-N-(4-bromo-phenyl)-benzamide (“RS-1”): 3-benzylsulfamoyl-4-bromo-benzoic acid (455 mg, 1.23 mmol) was dissolved in 5 mL of THF and 0.1 mL of DMF was added. To the reaction mixture oxalyl chloride (0.21 mL, 2.46 mmol) was added at room temperature. Reaction mixture was refluxed for 15 min, cooled and volatiles were removed under vacuum. Residue was re-dissolved in 5 mL of THF and 4-bromoaniline (254 mg, 1.48 mmol) in 1 mL of THF was added drop-wise followed by drop-wise addition of triethylamine (0.17 mL, 1.23 mmol). Reaction was stirred at room temperature for 2 h and 10 mL of ethyl acetate and 10 mL of water were added. Aqueous phase were further extracted with two portions of 15 mL of ethyl acetate. Organic phases were combined, washed with brine and dried with sodium sulfate. Solvents were evaporated and residue purified by preparative HPLC giving 257 mg (32%) of 3-benzylsulfamoyl-4-bromo-N-(4-bromo-phenyl)-benzamide as a white solid. ¹H-NMR (400 MHz, DMSO-d₆): δ 10.60 (s, 1H), 8.55 (t, J=6.1 Hz, 1H), 8.45 (d, J=2.1 Hz, 1H), 8.01 (dd, J=2.1 Hz, J=6.3 Hz, 1H), 7.95 (d, J=8.2 Hz, 1H), 7.75 (d, J=7 Hz, 2H), 7.57 (d, J=8.8 Hz, 2H), 7.22 (m, 5H), 4.15 (d, J=6.2 Hz, 2H). ¹³C-NMR (100 MHz, DMSO-d₆): δ 164.0, 140.7, 138.6, 137.8, 135.8, 134.5, 132.7, 132.0, 130.4, 128.5, 128.0, 127.6, 123.3, 122.9, 116.3, 46.5.

### Example 3

### Low Levels of RAD54B and RAD54L Expression Area Associated with Sensitivity to RS-1 in Immortalized Human Cells

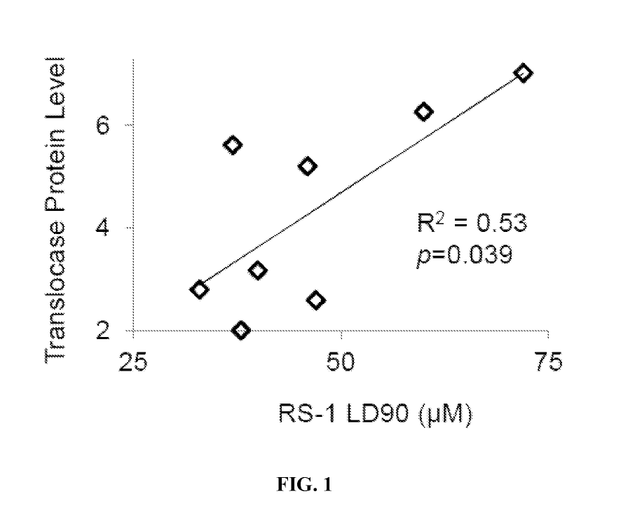

High levels of RAD51 overexpression render cells susceptible to the formation of toxic RAD51 complexes, particularly in cell types that harbor inadequate translocase activity (Shah, et al., 2010). The inventors examined whether malignant human cells with low/limiting levels of RAD54 translocase proteins would be hypersensitive to RS-1, a compound that increases the dNA binding activity of RAD51 (Jayathilaka, et al., 2008), using a panel of immortalized human cell lines. Whole cell levels for RAD51, RAD54L, and RAD54B proteins were measured by western blot, and the quantification for each cell line was normalized to levels in PC3 cells (Table 1). These relative protein levels were directly compared against RS-1 sensitivity (LD₉₀ values) by linear regression analysis. The factor most strongly associated with RS-1 LD₉₀ was RAD54B protein level (R²=0.33). By contrast, the association RS-1 LD₉₀ was considerably weaker for RAD51 and RAD54L protein levels (R²=0.04 and 0.19, respectively). A combined translocase protein expression level score was generated, which represents the sum of the RAD54B and RAD54L levels. Using this score, a significant correlation (R²=0.53, p=0.039) was observed between low translocase expression level and RS-1 sensitivity (FIG. 1).

### Methods

Western Blotting.

Whole cell protein extracts were separated via SDS PAGE and subjected to western blotting. Primary antibodies included protein A purified rabbit anti HsRAD51 (1:1000 dilution, gift of Akira Shinohara), RAD54L antibody (1:1000 dilution, 4E3/1 from Abcam), RAD54B antibody (1:1000 dilution, PA529881 from Thermo Scientific), mouse anti a tubulin (1:5000 dilution, Ab-2 from Fitzgerald). Secondary antibodies consisted of HRP-conjugated anti-rabbit IgG (1:1000 dilution, GE healthcare) and HRP-conjugated anti-mouse IgG (1:2000 dilution, GE healthcare).

Cell Survival Assays.

Cells were plated into 96-well tissue culture plates at a density of 300 cells per well in the presence or absence of RS-1 for 24 hours at 37° C., 5% CO₂. RS-1 was then removed, and cultures were allowed to grow for approximately one week to a 50-70% confluence. Average survival from six replicates was measured using CellGlo reagent (Promega), and error bars represent the standard error.

TABLE 1
Relative protein levels for a panel of human cell lines.

| Cell Type | RAD51 | RAD54L | RAD54B |
|---|---|---|---|
| PC3 | ≡1.0 | ≡1.0 | ≡1.0 |
| LNCaP | 0.4 | 0.9 | 1.9 |
| DU 145 | 0.6 | 0.3 | 5.3 |
| COLO 205 | 1.7 | 1.1 | 1.4 |
| MCF-7 | 1.0 | 1.7 | 5.3 |
| HEK-293 | 3.5 | 2.2 | 4.1 |
| U2OS | 4.5 | 2.6 | 2.6 |
| MDA-MB-231 | 3.4 | 2.2 | 0.9 |

### Example 4

### Sensitivity to RS-1 is Dependent on RAD51 and RAD54B/RAD54L Translocases

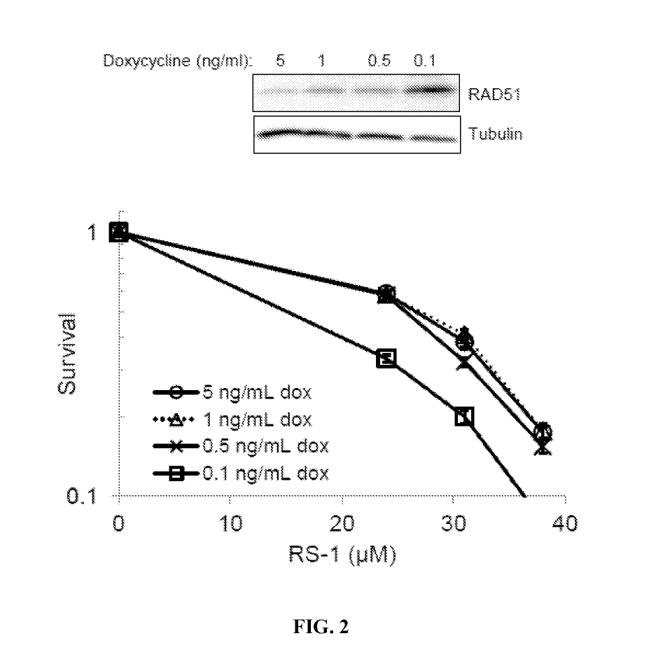

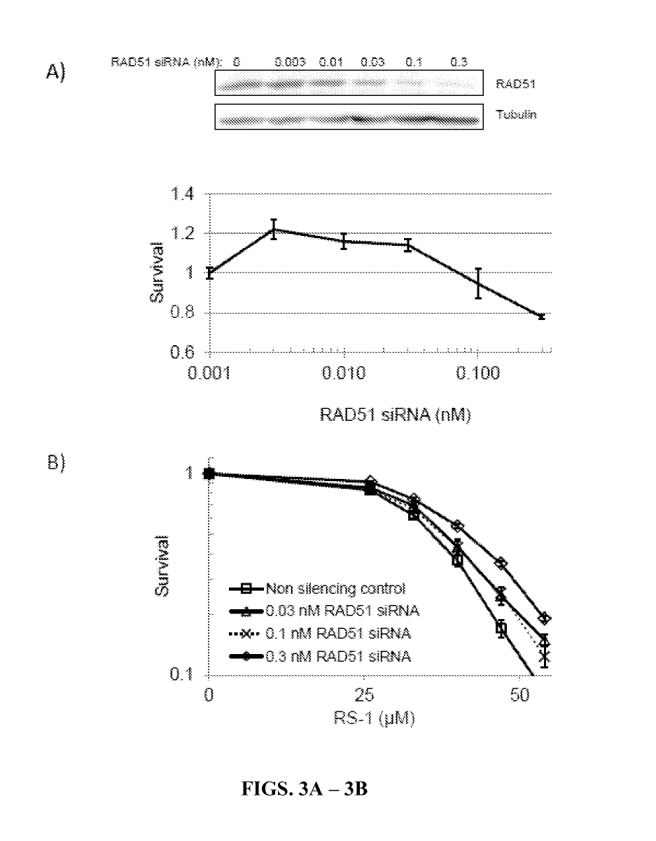

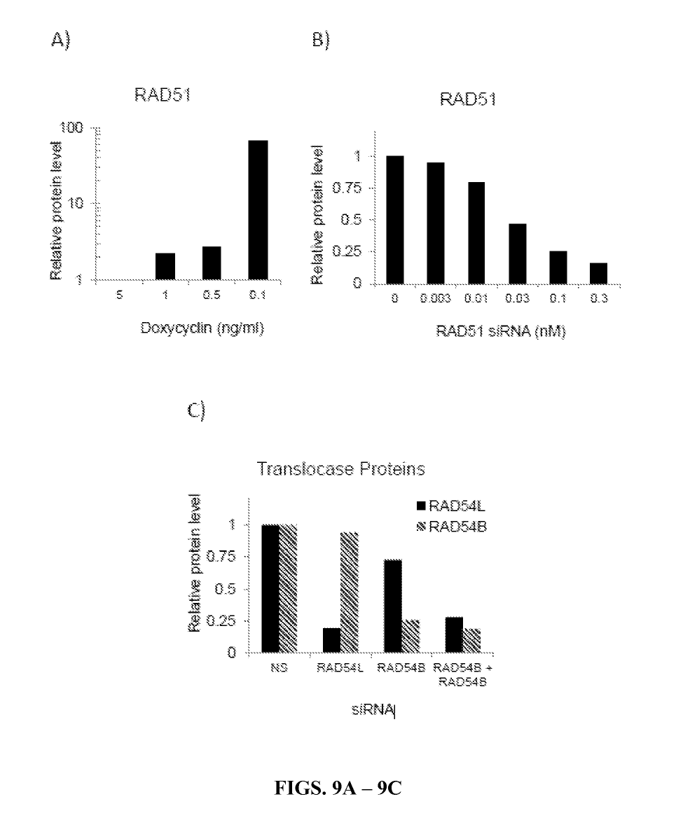

To confirm that RS-1 toxicity is directly related to RAD51 and translocases protein levels, these proteins were experimentally manipulated in cells. First, RAD51 was overexpressed in human fibrosarcoma HT1080 cells carrying a doxycycline-repressible RAD51 transgene. Consistent with published data (Flygare, et al., 2001) the removal of doxycycline from media generated high levels of RAD51 expression, reaching a 12.7-fold increase with 0.1 ng/ml doxycycline relative to 5 ng/ml doxycycline (FIG. 2, see quantifications of western blots in FIG. 9). Cells with the highest RAD51 expression levels were significantly more sensitive to RS-1. Next, the inventors tested whether knocking down RAD51 levels with RNAi would ameliorate RS-1 toxicity. The prostate cancer cell line PC3 was selected for these experiments, because the low LD₉₀ to RS-1 suggests a particular susceptibility of PC3 to forming toxic RAD51 complexes. Interestingly, modest depletion of RAD51 levels (5% to 50%) increased the viability of PC3 cells by about 20% in the absence of any other treatment (FIG. 3). When RAD51 siRNA was combined with RS-1 treatment, the RAD51 depletion generated significant protection from RS-1 induced toxicity. Taken together, these results suggest that the level of RAD51 in PC3 cells limits survival, a likely consequence of toxic RAD51 complexes. Correspondingly, RAD51 depletion enhances survival, while stimulation of RAD51 complex formation by RS-1 reduces survival.

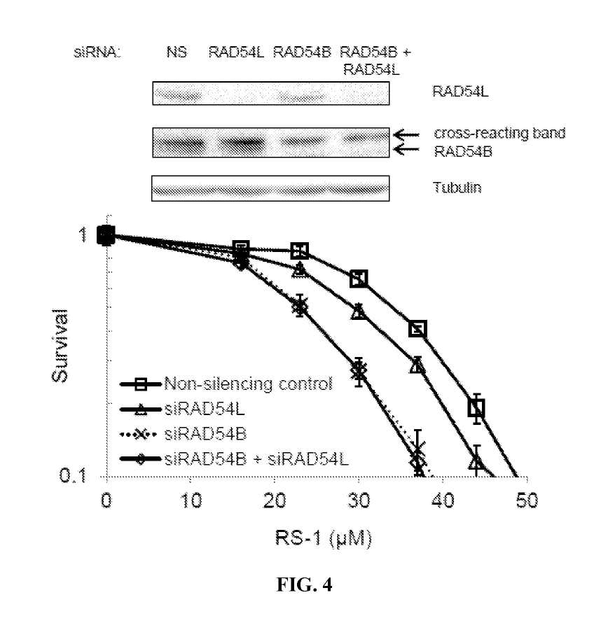

The ability of translocase proteins to ameliorate RS-1 induced toxicity was tested by knocking down RAD54B and RAD54L with RNAi (FIG. 4) in PC3 cells. The knockdown of either translocase significantly sensitized PC3 cells to RS-1 toxicity, though the impact of RAD54B was larger than that of RAD54L. Combined knockdown of both RAD54 translocases did not generate more RS-1 sensitization than RAD54B siRNA alone, suggesting RAD54B has more activity in ameliorating RAD51-dependent toxicity, at least in the context of RS-1 treatment.

### Methods

Knockdown of RAD51, RAD54L, and RAD54B.

The RAD51 siRNA and the All-Stars negative control siRNA (NS) were ordered from Qiagen. RAD54B siRNAs were ordered from Invitrogen (Stealth). The RAD54L siRNA cocktail was ordered from Santa Cruz (sc-36362). All siRNAs were transfected using RNAiMax as per manufacturer's instructions (Invitrogen). Briefly, 2.0×10⁵ cells were plated in 6 well dishes containing siRNA complexes to achieve the desired final concentration of siRNAs. The RAD54B and RAD54L siRNAs consisted of a cocktail of three independent siRNAs. The concentration of siRNAs transfected were 25 nM for RAD54L and 50 nM RAD54B. Co-depletion of RAD54L and RAD54B was performed by transfecting cells simultaneously with both siRNAs. At 48 hours post transfection, cells were harvested for cell survival assays and western blotting as described. The target sequences for siRNA depletion are as follows:

RAD51
(SEQ ID NO. 1)
5′ AAGCTGAAGCGAGTTCGCCA
RAD54B-1
(SEQ ID NO. 2)
5′ CCTCATTAGCCTTTCTTGTGAGAAA
RAD54B-2
(SEQ ID NO. 3)
5′ GCTAGGAAGTGAAAGGATCAAGATA
RAD54B-3
(SEQ ID NO. 4)
5′ GACATTGGAAGAGGCATTGGTTATA
RAD54L-A
(SEQ ID NO. 5)
5′ GATCTGCTTGAGTATTTCA
RAD54L-B
(SEQ ID NO. 6)
5′ CCGTAGCAGTGACAAAGTA
RAD54L-C
(SEQ ID NO. 7)
5′ GAACCCAGCCAATGATGAA

### Example 5

### RS-1 Treatment Results in the Accumulation of RAD51 Complexes on Undamaged Chromatin in PC3 Cells

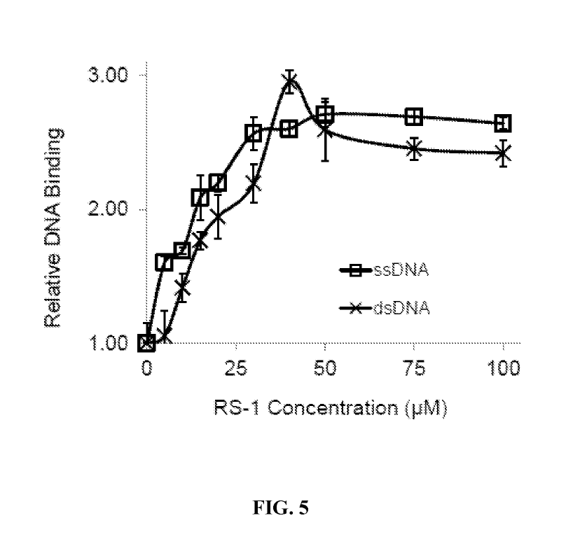

Some cancer cells that strongly overexpress RAD51 are known to develop spontaneous RAD51 nuclear complexes (Raderschall, et al., 2002). Therefore, such cells may be especially susceptible to RS-1 mediated RAD51 complexes on undamaged dsDNA, since RS-1 stimulates the binding of RAD51 to both ssDNA and dsDNA (FIG. 5). PC3 prostate cancer cells and normal primary human fibroblasts (MRC-5) were treated with RS-1 and examined by immunofluorescence microscopy. To determine whether RAD51-staining structures represented sites of DNA repair vs. non-damage-associated sites, nuclei were counterstained for RPA, which forms punctuate sub-nuclear foci specifically in response to DNA damage at sites that colocalize with damage-induced RAD51 foci (Golub, et al., 1998).

At baseline with no treatment, 4.6% of PC3 cells exhibited >10 discrete RAD51 foci/nucleus, whereas 0% of the non-cancerous control cells (MRC-5) exhibited >10 RAD51 foci/nucleus (FIG. 6). After treatment with RS-1, this difference became markedly more obvious. Specifically, 43% of PC3 nuclei exhibited >10 foci/nucleus after RS-1 treatment, while again no MRCS nuclei exhibited >10 RAD51 foci/nucleus. Neither cell type exhibited >10 RPA foci/nucleus in any cells after RS-1 treatment. As a control, both cell types were also examined after ionizing radiation, and as expected PC3 and MRCS cells exhibited >10 foci/nucleus with both RAD51 (72% and 29%, respectively) and RPA staining (35% and 21%, respectively). This indicates that RAD51 levels are not limiting for RAD51 focus formation in MRCS cells. These results suggest that RS-1 treatment specifically leads to the accumulation of RAD51 foci in PC3 and not MRC-5 cells, via a mechanism that is independent of DNA damage.

### Methods

Microscopy to Detect RAD51 and RPA Foci.

Cells were grown on coverslips and treated with RS-1 or radiation as indicated. They were subsequently fixed with 3% paraformaldahyde/3.4% sucrose, and permeabilized with a standard buffer (20 mM HEPES pH 7.4, 0.5% TritonX-100, 50 mM NaCl, 3 mM MgCl₂, 300 mM sucrose). Slides were then stained with a rabbit polyclonal HsRAD51 antibody (1:2500 dilution) and/or a mouse monoclonal RPA antibody (1:1000 dilution, Ab-2 from CalBioChem), followed by Alexa 488-conjugated goat anti-rabbit and Alexa 594-conjugated goat anti-mouse secondary antibodies (Invitrogen, both 1:2000 dilution). Slides were viewed using a Zeiss Axio Imager.M1 microscope that allows high-resolution detection of foci throughout the entire nuclear volume. Images were recorded at a single representative focal plane using a CCD camera. For each experimental condition, 50 randomly selected nuclei were quantified using NIH Image software. For the purpose of RPA quantification, cells with diffuse RPA staining patterns, including S-phase cells, were excluded from the analysis as it is difficult to obtain reliable focus counts in these cells.

Measurements of RAD51 Binding to DNA.

Experiments were performed as previously described with some modifications (Budke, et al., 2012). Briefly, 75 nM purified human RAD51 protein was incubated with various concentrations of RS-1 in FP reaction buffer at 37° C. for 40 minutes. FP reaction buffer consisted of 20 mM Hepes pH 7.5, 10 mM MgCl₂, 0.25 μM BSA, 2% glycerol, 30 mM NaCl, 4% DMSO, 0.1 mM tris(2-carboxyethyl)phosphine (TCEP), and 2 mM ATP. Fluorescently tagged DNA substrate was then added to a final concentration of 100 nM (nucleotide concentration for ssDNA or base pair concentration for dsDNA) and incubated at 37° C. for another 40 minutes. DNA substrates consisted of either an Alexa Fluor 488-labeled oligo-dT 45-mer, a fluorescein-labeled ssDNA oligonucleotide (DHD162-CD-CF), or a fluorescein-labeled dsDNA double hairpin (DHD162) which were previously described (Budke, et al., 2013). Fluorescence polarization measurements were obtained as previously described (Budke, et al., 2012). The indicated concentrations of RAD51 and compounds reflect their concentrations in the final 50 IA reaction mixture.

### Example 6

### RS-1 Generates Anti-Tumor Responses in an Animal Model

An in-vivo tumor model was used to further test the concept of RAD51 stimulation as a cancer treatment. Subcutaneous xenograft PC3 tumors were induced in the hind limbs of athymic nude mice, and the mice were subsequently treated with daily intra-peritoneal injections of RS-1 for five consecutive days. The daily dose administered to the mice was designed to yield an idealized concentration of 300 μM within the aqueous compartment of a mouse, based on an assumption of homogenous distribution across a 21 gm animal that is composed of 70% water.

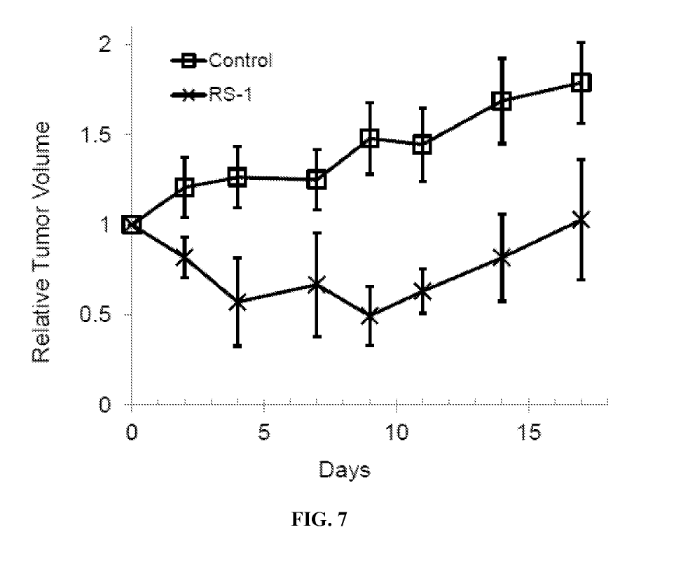

Treatment with RS-1 generated significant anti-tumor responses relative to the vehicle-alone control mice, whose tumors all progressively grew (FIG. 7). 43% of tumors (3 of 7) in the RS-1 group completely disappeared after treatment and never regrew during a two month observation period. The remaining tumors in the RS-1 treated group did eventually regrow; however, RS-1 treatment generated a >2 week delay in tumor regrowth relative to the vehicle-alone control. RS-1 treatment was relatively well tolerated, with no toxic deaths observed. Mice treated with RS-1 experienced a transient weight loss of about 10% during the treatment week; however, they completely regained this weight in the post-treatment period and demonstrated no other overt signs of drug toxicity. This experiment was repeated, and the result reproduced.

### Methods

Mouse Tumor Experiments.

Xenograft tumors were induced in the hind limbs of athymic nude mice by subcutaneous injection of 1×10⁶ PC3 cells, and tumors were allowed to grow to an average volume of about 50 mm³. Mice were then were randomized into treatment groups, each consisting of 7 mice. Peritoneal administrations of RS-1 were delivered in 200 μl of a vehicle solutions, which consisted of 30% DMSO, 35% PEG-400, 35% PBS. Tumor measurements were taken 3 times per week with a caliper and expressed as tumor volume, which was approximated from the product of width×length×height×0.5. Displayed points denote the median fractional tumor volume, and error bars denote standard error.

All of the methods and apparatuses disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Bello, et al., Biochem Pharmacol. 63: 1585-8, 2002.
Bishop, et al., Cell. 79: 1081-92, 1994.
Brown & Holt, Mol Carcinog. 48: 105-9, 2009.
Budke, et al., Nucleic Acids Res. 41: e130, 2013.
Budke, et al., J Med Chem. 56: 254-63, 2013.
Budke, et al., Nucleic Acids Res. 40: 7347-57, 2012.
Connell, et al., Int J Oncol. 28: 1113-9, 2006.
Flygare, et al., Exp Cell Res. 268: 61-9, 2001.
Golub, et al., Nucleic Acids Res. 26: 5388-93, 1998.
Haaf, et al., Proc Natl Acad Sci USA. 92: 2298-302, 1995.
Han et al., Cancer Res., 62:2890-2896, 2002.
Hansen, et al., Int J Cancer. 105: 472-9, 2003.
Henning and Sturzbecher, Toxicology, 193:91-109, 2003.
Hine, Proc Natl Acad Sci USA. 105: 20810-5, 2008.
Hiramoto et al., Oncogene. 18(22):3422-26, 1999.
Jayathilaka, et al., Proc Natl Acad Sci USA. 105: 15848-53, 2008.
Kim, et al., Nucleic Acids Res. 29: 4352-60, 2001.
Klein, DNA Repair. 7: 686-93, 2008.
Lee, et al., DNA Repair. 8: 170-81, 2009.
Li et al., Carcinogenesis. 34(7):1536-42, 2013.
Liu, et al., Mol Cell. 1: 783-93, 1998.
Maacke, et al., Int J Cancer. 88: 907-13, 2000a.
Maacke et al., J. Cancer Res. Clin. Oncol., 128:219-222, 2002.
Maacke et al., Oncogene, 19:2791-2795, 2000b.
Martin, et al., Cancer Res. 67: 9658-65, 2007.
Mason, et al., RAD54-family translocases promote proper chromosome segregation, genome stability and viability of human tumor cells expressing high levels of the strand exchange protein RAD51. University of Chicago; 2013.
Mitra, et al., Histopathology. 55: 696-704, 2009.
Qiao, et al., Br J Cancer. 93: 137-43, 2005.
Raderschall, et al., Cancer Res. 62: 219-25, 2002.
Richardson, et al., Oncogene. 23: 546-53, 2004.
Shah, et al., Mol Cell. 39: 862-72, 2010.
Slupianek, et al., Mol Cell. 8: 795-806, 2001.
Solinger, et al., Mol Cell. 10: 1175-88, 2002.
Takata, et al., Mol Cell Biol. 21: 2858-66, 2001.
Takenaka, et al., Int J Cancer. 121: 895-900, 2007.
Tebbs, et al., Proc Natl Acad Sci USA. 92: 6354-8, 1995.
Thompson & Schild, Mutat Res. 477: 131-53, 2001.
Vispe, et al., Nucleic Acids Res. 26: 2859-64, 1998.

1. A method of killing or inhibiting the growth of cells comprising contacting the cells with a composition comprising an amount of a RAD51 stimulator effective to kill or inhibit the growth of the cells.

2. The method of claim 1, wherein the RAD51 stimulator is a compound having the formula (VIIa): 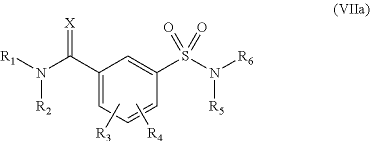
wherein:
R₁ is hydrogen, alkyl, aryl or aralkyl;
R₂ is alkyl, aryl or aralkyl;
X is O or S;
R₃ is hydrogen, halogen, alkyl or alkoxy;
R₄ is hydrogen, halogen, alkyl or alkoxy;
R₅ is hydrogen, alkyl, aryl, or aralkyl; and
R₆ is hydrogen, alkyl, aryl or aralkyl.

3. The method of claim 2, wherein R₃ is substituted at the 4 position and R₄ is substituted at the 6 position.

4. The method of claim 3, wherein the halogen of R₃ and R₄ are both chloride or bromide.

5. The method of claim 3, wherein R₃ is hydrogen and R₄ is either chloride or bromide.

6. The method of claim 3, wherein R₄ is hydrogen and R₃ is either chloride or bromide.

7. The method of claim 3, wherein R₃ is hydrogen and R₄ is methyl.

8. The method of claim 3, wherein R₃ is hydrogen and R₄ is methoxy.

9. The method of any of claims 2 through 8, wherein R₁ is: 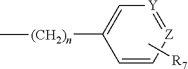
wherein:
n is 0-6;
Y is C or N;
Z is C or N; and
R₇ is hydrogen, halogen, alkyl, alkoxy or carboxy.

10. The method of claim 9, wherein Y and Z are both C and R₇ is substituted at the 2 or 4 position.

11. The method of claim 10, wherein R₇ is a chloride or bromide.

12. The method of claim 10, wherein R₇ is methyl.

13. The method of claim 10, wherein R₇ is methoxy.

14. The method of any of claims 2 through 13, wherein R₆ is: 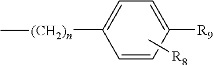
wherein:
n is 0-6;
R₈ is hydrogen or alkyl; and
R₉ is hydrogen, halogen or alkyl.

15. The method of claim 14, wherein R₈ is methyl and substituted at the 2 or 3 position.

16. The method of claim 15, wherein R₉ is methyl.

17. The method of claim 14, wherein R₈ is hydrogen and the halogen of R₉ is bromide.

18. The method of claim 1, wherein the RAD51 stimulator is a compound having the formula (VIIb): 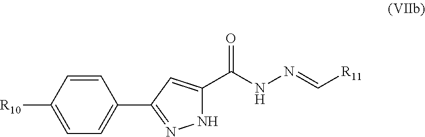
wherein:
R₁₀ is halogen or alkoxy; and
R₁₁ is aryl.

19. The method of claim 18, wherein R₁₀ is chloride.

20. The method of claim 18, wherein R₁₀ is methoxy or ethoxy.

21. The method of claim 18 through 20, wherein R₁₁ is: 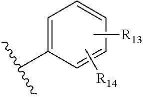
wherein:
R₁₃ is hydroxyl or methoxy; and
R₁₄ is hydroxyl.

22. The method of claim 21, wherein R₁₃ is substituted at the 4 position and R₁₄ is substituted at the 2 or 3 position.

23. The method of claim 1, wherein the RAD51 stimulator is a compound having the formula (VIIc): 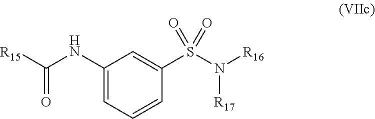
wherein:
R₁₅ is C₁-C₁₀ alkyl,
R₁₆ is aryl; and
R₁₇ is hydrogen.

24. The method of claim 23, wherein R₁₅ is iso-butyl.

25. The method of claim 23, wherein R₁₅ is 4-bromophenyl.

26. The method of claim 1, wherein the RAD51 stimulator is a compound having the following formula:

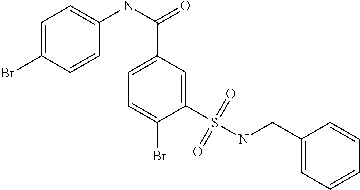 [SMILES: O=C(Nc1ccc(Br)cc1)c1ccc(Br)c(S(=O)(=O)NCc2ccccc2)c1]

27. The method of any of claims 1 to 26, wherein the cells have an increased sensitivity to the RAD51 stimulator relative to a control level of sensitivity.

28. The method of any of claims 1 to 27, wherein the cells are determined to have an increased sensitivity to the RAD51 stimulator relative to a control level of sensitivity.

29. The method of any of claims 1 to 28, wherein the cells express an increased level of RAD51 relative to a control level.

30. The method of any of claims 1 to 29, wherein the cells have been determined to express an increased level of RAD51 relative to a control level.

31. The method of any of claims 1 to 30, wherein the cells have a decreased activity or expression level of RAD54B, RAD54L, or both, relative to a control level.

32. The method of any of claims 1 to 31, wherein the cells have been determined to have a decreased activity or expression level of RAD54B, RAD54L, or both, relative to a control level.

33. The method of any of claims 1 to 32, wherein the cells are in cell culture.

34. The method of any of claims 1 to 33, wherein the cells are in a patient's body.

35. The method of any of claims 1 to 34, wherein the cells are cancer cells.

36. The method of any of claims 1 to 35, wherein the cells are in a tumor.

37. The method of any of claims 1 to 36, wherein the composition comprises 20 to 80 μM of RAD51 stimulator.

38. The method of any of claims 1 to 37, wherein the cells are not exposed to a substantial amount of any DNA damaging agent.

39. The method of any of claims 1 to 37, further comprising contacting the cells with a DNA damaging agent.

40. The method of claim 39, wherein the DNA damaging agent comprises one or more of 5-FU, capecitabine, S-1, ara-C, 5-AC, dFdC, a purine antimetabolite, gemcitabine hydrochlorine, pentostatin, allopurinol, 2F-ara-A, hydroxyurea, sulfur mustard, mechlorethamine, melphalan, chlorambucil, cyclophosphamide, ifosfamide, thiotepa, AZQ, mitomycin C, dianhydrogalactitol, dibromoducitol, busulfan, a nitrosourea, procarbazine, decarbazine, rebeccamycin, an anthracyclin, an anthracyclin analog, a non-intercalating topoisomerase inhibitor, podophylotoxin, bleomycin, pepleomycin, cisplatin, trans analog of cisplatin, carboplatin, iproplatin, tetraplatin and oxaliplatin, camptothecin, topotecan, irinotecan, SN-38, UV radiation, IR radiation, α-, β-, and γ-radiation.

41. The method of any of claims 1 to 40, further comprising contacting the cells with a RAD54 inhibitor.

42. The method of claim 41, wherein the RAD54 inhibitor comprises streptonigrin.

43. A method of selectively killing or inhibiting the growth of cancer cells in a subject comprising administering to the subject a pharmaceutically acceptable composition comprising an amount of RAD51 stimulator effective to selectively kill or inhibit the growth of the cancer cells.

44. The method of claim 43, wherein the RAD51 stimulator is a compound having the formula (VIIa): 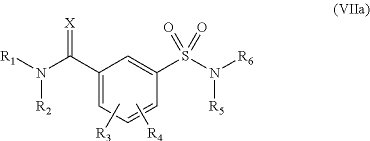
wherein:
R₁ is hydrogen, alkyl, aryl or aralkyl;
R₂ is alkyl, aryl or aralkyl;
X is O or S;
R₃ is hydrogen, halogen, alkyl or alkoxy;
R₄ is hydrogen, halogen, alkyl or alkoxy;
R₅ is hydrogen, alkyl, aryl, or aralkyl; and
R₆ is hydrogen, alkyl, aryl or aralkyl.

45. The method of claim 44, wherein R₃ is substituted at the 4 position and R₄ is substituted at the 6 position.

46. The method of claim 45, wherein the halogen of R₃ and R₄ are both chloride or bromide.

47. The method of claim 45, wherein R₃ is hydrogen and R₄ is either chloride or bromide.

48. The method of claim 45, wherein R₄ is hydrogen and R₃ is either chloride or bromide.

49. The method of claim 45, wherein R₃ is hydrogen and R₄ is methyl.

50. The method of claim 45, wherein R₃ is hydrogen and R₄ is methoxy.

51. The method of any of claims 44 through 50, wherein R₁ is: 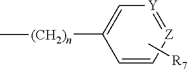
wherein:
n is 0-6;
Y is C or N;
Z is C or N; and
R₇ is hydrogen, halogen, alkyl, alkoxy or carboxy.

52. The method of claim 51, wherein Y and Z are both C and R₇ is substituted at the 2 or 4 position.

53. The method of claim 52, wherein R₇ is a chloride or bromide.

54. The method of claim 52, wherein R₇ is methyl.

55. The method of claim 52, wherein R₇ is methoxy.

56. The method of any of claims 44 through 55, wherein R₆ is: 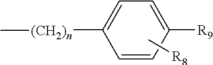
wherein:
n is 0-6;
R₈ is hydrogen or alkyl; and
R₉ is hydrogen, halogen or alkyl.

57. The method of claim 56, wherein R₈ is methyl and substituted at the 2 or 3 position.

58. The method of claim 57, wherein R₉ is methyl.

59. The method of claim 56, wherein R₈ is hydrogen and the halogen of R₉ is bromide.

60. The method of claim 43, wherein the RAD51 stimulator is a compound having the formula (VIIb): 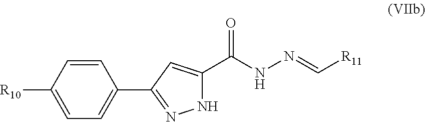
wherein:
R₁₀ is halogen or alkoxy; and
R₁₁ is aryl.

61. The method of claim 60, wherein R₁₀ is chloride.

62. The method of claim 60, wherein R₁₀ is methoxy or ethoxy.

63. The method of claim 60 through 62, wherein R₁₁ is: 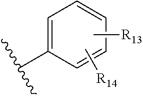
wherein:
R₁₃ is hydroxyl or methoxy; and
R₁₄ is hydroxyl.

64. The method of claim 63, wherein R₁₃ is substituted at the 4 position and R₁₄ is substituted at the 2 or 3 position.

65. The method of claim 43, wherein the RAD51 stimulator is a compound having the formula (VIIc): 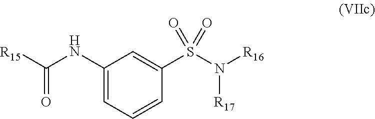
wherein:
R₁₅ is C₁-C₁₀ alkyl,
R₁₆ is aryl; and
R₁₇ is hydrogen.

66. The method of claim 66, wherein R₁₅ is iso-butyl.

67. The method of claim 66, wherein R₁₅ is 4-bromophenyl.

68. The method of claim 43, wherein the RAD51 stimulator is a compound having the following formula:

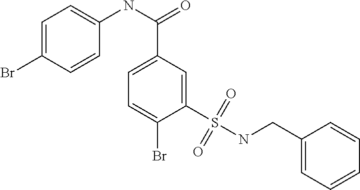 [SMILES: O=C(Nc1ccc(Br)cc1)c1ccc(Br)c(S(=O)(=O)NCc2ccccc2)c1]

69. The method of any of claims 43 to 68, wherein the subject has cancer of the lung, liver, skin, eye, brain, gum, tongue, hematopoietic system or blood, head, neck, breast, pancreas, prostate, kidney, bone, testicles, ovary, cervix, gastrointestinal tract, lymph system, small intestine, colon, or bladder.

70. The method of any of claims 43 to 69, wherein the cancer cells are in a tumor.

71. The method of claim 70, wherein the composition comprises an amount of RAD51 stimulator effective to shrink or inhibit the growth of the tumor.

72. The method of any of claims 43 to 71, wherein the cancer cells have an increased sensitivity to the RAD51 stimulator relative to a control level of sensitivity.

73. The method of any of claims 43 to 72, wherein the cancer cells have been determined to have an increased sensitivity to the RAD51 stimulator relative to a control level of sensitivity.

74. The method of any of claims 43 to 73, wherein the cancer cells express an increased level of RAD51 relative to a control level.

75. The method of any of claims 43 to 74, wherein the cancer cells have been determined to express an increased level of RAD51 relative to a control level.

76. The method of any of claims 43 to 75, wherein the cancer cells have a decreased activity or expression level of RAD54B, RAD54L, or both, relative to a control level.

77. The method of any of claims 43 to 76, wherein the cancer cells have been determined to have a decreased activity or expression level of RAD54B, RAD54L, or both, relative to a control level.

78. The method of any of claims 43 to 77, wherein the subject is administered a dose of 50 to 150 mg/kg of the RAD51 stimulator.

79. The method of any of claims 43 to 78, wherein the subject is administered a dose of 110 mg/kg.

80. The method of any of claims 43 to 79, wherein the RAD51 stimulator is present in the blood of the subject in a concentration of 250 to 350 μM.

81. The method of any of claims 43 to 80, wherein the RAD51 stimulator is present in the blood of the subject in a concentration of 300 μM.

82. The method of any of claims 43 to 81, wherein the subject is not administered a substantial amount of any DNA damaging agent within three days of administering to the subject the RAD51 stimulator.

83. The method of any of claims 43 to 81, wherein the subject is not exposed to a substantial amount of any DNA damaging agent within seven days of administering the RAD51 stimulator to the subject.

84. The method of any of claims 43 to 81, wherein the subject is not exposed to a substantial amount of any DNA damaging agent after administering the RAD51 stimulator to the subject.

85. The method of any of claims 43 to 84, wherein the subject is not administered a DNA damaging agent as part of a combination therapy with the RAD51 stimulator.

86. The method of any of claims 43 to 85, wherein the RAD51 stimulator is administered to the subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularly, mucosally, intrapericardially, intraumbilically, intraocularally, orally, topically, locally, by inhalation, by injection, by infusion, by continuous infusion, by localized perfusion bathing target cells directly, via a catheter, or via a lavage.

87. The method of any of claims 43 to 86, wherein the RAD51 stimulator is administered to the patient multiple times.

88. The method of any of claims 43 to 87, wherein the subject is administered an additional cancer therapy.

89. The method of any of claims 43 to 81, wherein the subject is administered a DNA damaging agent as part of a combination therapy with the RAD51 stimulator.

90. The method of any of claims 43 to 81, wherein the subject is administered a DNA damaging agent within three days of administering to the subject the RAD51 stimulator.

91. The method of any of claims 43 to 81, wherein the subject is administered a DNA damaging agent within seven days of administering to the subject the RAD51 stimulator.

92. The method of any of claims 43 to 81, wherein the subject is administered a substantial amount of a DNA damaging agent after administering the RAD51 stimulator to the subject.

93. The method of any of claims 89 to 92, wherein the DNA damaging agent comprises one or more of 5-FU, capecitabine, S-1, ara-C, 5-AC, dFdC, a purine antimetabolite, gemcitabine hydrochlorine, pentostatin, allopurinol, 2F-ara-A, hydroxyurea, sulfur mustard, mechlorethamine, melphalan, chlorambucil, cyclophosphamide, ifosfamide, thiotepa, AZQ, mitomycin C, dianhydrogalactitol, dibromoducitol, busulfan, a nitrosourea, procarbazine, decarbazine, rebeccamycin, an anthracyclin, an anthracyclin analog, a non-intercalating topoisomerase inhibitor, podophylotoxin, bleomycin, pepleomycin, cisplatin, trans analog of cisplatin, carboplatin, iproplatin, tetraplatin and oxaliplatin, camptothecin, topotecan, irinotecan, SN-38, UV radiation, IR radiation, α-, β-, and γ-radiation.

94. The method of any of claims 43 to 93, further comprising administering to the subject a RAD54 inhibitor.

95. The method of claim 94, wherein the RAD54 inhibitor comprises streptonigrin.

96. A method of treating cancer in a patient comprising administering an effective amount of a RAD51 stimulator after determining that the cancer has increased sensitivity to the RAD51 stimulator relative to a control level of sensitivity.

97. The method of claim 96, further comprising measuring the expression or activity level of RAD51, RAD54B, and/or RAD54L, in the cancer and comparing it to a control level.


SEQUENCE LISTING (7 sequences)

SEQ ID NO: 1 - DNA, 20 bp, Artificial Sequence
  Synthetic Primer
aagctgaagcgagttcgcca

SEQ ID NO: 2 - DNA, 25 bp, Artificial Sequence
  Synthetic Primer
cctcattagcctttcttgtgagaaa

SEQ ID NO: 3 - DNA, 25 bp, Artificial Sequence
  Synthetic Primer
gctaggaagtgaaaggatcaagata

SEQ ID NO: 4 - DNA, 25 bp, Artificial Sequence
  Synthetic Primer
gacattggaagaggcattggttata

SEQ ID NO: 5 - DNA, 19 bp, Artificial Sequence
  Synthetic Primer
gatctgcttgagtatttca

SEQ ID NO: 6 - DNA, 19 bp, Artificial Sequence
  Synthetic Primer
ccgtagcagtgacaaagta

SEQ ID NO: 7 - DNA, 19 bp, Artificial Sequence
  Synthetic Primer
gaacccagccaatgatgaa